(19) United States
(12) Patent Application Publication
(10) Pub. No.: US 2011/0311558 A1
(43) Pub. Date: Dec. 22, 2011

(54) Recombinant Bone Marrow Stromal Antigen-2 in the Treatment of Autoimmune Diseases

(75) Inventors: Wei Cao, Bellaire, TX (US); Yong-Jun Liu, Pearland, TX (US)

(73) Assignee: THE BOARD OF REGENTS OF THE UNIVERSITY OF TEXAS SYSTEM, Austin, TX (US)

(21) Appl. No.: 13/131,514

(22) PCT Filed: Dec. 1, 2009
(86) PCT No.: PCT/US09/66246

§ 371 Date: Sep. 6, 2011

Related U.S. Application Data
(60) Provisional application No. 61/118,948, filed on Dec. 1, 2008.

(51) Int. Cl. A61K 39/395; A61P 17/06; C07K 19/00; A61P 37/06; A61K 39/00; C07K 14/705

(52) U.S. Cl. 424/178.1; 424/184.1; 424/185.1; 530/395

(57) ABSTRACT

Methods, compositions and kits are disclosed for inhibiting interferon production and modulating immune responses, particularly an autoimmune response. In certain embodiments, the methods involve administering an effective amount of a BST2 protein or a nucleic acid encoding an BST2 protein to treat an autoimmune disease or disorder.

The present application claims benefit of priority to U.S. Provisional Application Ser. No. 61/118,948 filed Dec. 1, 2009, the entire contents of which are hereby incorporated by reference.

This invention was made with U.S. government support under grant A1074809 from the National Institutes of Health. The government has certain rights in the invention.

### BACKGROUND OF THE INVENTION

I. Field of the Invention

The present invention relates to methods and compositions for modulating an immune response, in particular, an autoimmune response. More specifically, the invention discloses the use of a BST2 protein agent to suppress an immune response, to reduce the production of interferon, and/or to treat an autoimmune disease.

II. Background and Description of Related Art

The immune system is the body's primary defense against invading organisms, such as bacteria, viruses or parasites, and diseases caused by abnormal growth of the body's own tissues (i.e., tumors). Normally, the immune system is able to distinguish the body's normal tissues, or “self”, from foreign or cancerous tissue, or “non-self”. The loss of recognition of a particular tissue as self, and the subsequent immune response directed against that tissue are typically referred to as an “autoimmune response.” Once healthy tissues and organs have become indistinguishable from pathogens or cancer cells to the immune system, the result is most often a serious and debilitating destruction of tissue and subsequent chronic disease. Thus, neutralizing or inhibiting an autoimmune response is desirable in patients with autoimmune diseases.

The treatment of autoimmunity is important considering the significant rise in the incidence of the nearly one hundred known forms of autoimmune disease. As a group, autoimmune diseases are now the second most common chronic illness in the US, and the third leading cause of Social Security disability behind heart disease and cancer. Nearly 24 million Americans suffer from some form of autoimmunity, and these diseases have recently become the eighth leading cause of death among women in the US, shortening the average patient's lifespan by fifteen years. As autoimmune disease often involves long-term illness and disability, the economic burden is also substantial, with autoimmune diseases representing a yearly healthcare burden of more than $120 billion.

Many autoimmune diseases feature an abnormal level of or responsiveness to interferons. Interferons are glycoproteins produced by a variety of cells (interferon-producing cells, or IPCs) in response to challenges by foreign (or non-self) agents. As proteins, interferons are classified as cytokines, and their expression is upregulated by the JAK-STAT signaling pathway. Interferon production by most IPCs is actuated by the presence of double-stranded RNA, a key indicator of viral infection. The normal activities of interferons include inhibiting viral replication within host cells, activating natural killer cells and macrophages, increasing antigen presentation to lymphocytes, and inducing the resistance of host cells to viral infection. It follows that uncontrolled production of interferons, or production of interferons in the absence of an invader or tumor cell, can result in a range of abberant responses by the immune system. Thus, a means of reducing or inhibiting the production of interferons by IPCs may represent an advantageous strategy in treating autoimmune disease.

Currently available treatments for many forms of autoimmune disease are inadequate, expensive, or are associated with dangerous side effects. Since most treatment regimens must be carried out for long periods of time, these drawbacks have an even greater impact on their potential as therapies for chronic illness. Well-established agents in the treatment of many autoimmune diseases, such as corticosteroids and antimetabolites, are incompatible with commonly used drugs, cannot be administered safely to individuals with certain common illnesses, and can have both short- and long-term health implications. More recently developed therapies, such as TNF inhibitors, still produce serious side-effects and, because they are given by intravenous infusion, can become prohibitively expensive during the course of treatment.

In consideration of currently available treatments for autoimmune diseases, their increased incidence, and their degenerative and often life-threatening consequences, there continues to be an unmet need for methods and compositions for treating autoimmune disease.

### SUMMARY OF THE INVENTION

The present invention overcomes limitations in the prior art by providing methods and compositions for modulating an immune response, in particular, an autoimmune response, by inhibiting the production of interferon in plasmacytoid dendritic cells. The invention includes the use of a BST2 protein agent to suppress the production of interferon and treat autoimmune diseases.

The present invention provides a method of treating an autoimmune disease comprising identifying a subject having or suspected of having an autoimmune disease, and administering to the subject a therapeutically effective amount of a pharmaceutical composition comprising a BST2 protein agent. In some embodiments, the pharmaceutical composition can further comprise one or more pharmaceutically acceptable excipients.

In certain embodiments, the invention provides a method of treating an autoimmune disease in which the BST2 protein agent is administered to a subject in an amount which is sufficient to inhibit in a cell of the subject, one or more of type I interferon production, inflammatory cytokine production, and inflammatory chemokine production. In other embodiments, type I interferon production is inhibited in a plasmacytoid dendritic cell of the subject.

In a particular aspect, the BST2 protein agent inhibits in a subject one or more aspects of an autoimmune response including, without limitation, a mixed leukocyte reaction; a macrophage response; a natural killer reaction, a lymphocyte activation, production of type I interferon, production of autoantibodies.

In some embodiments, the invention provides a method of treating an autoimmune disease in which the BST2 protein agent inhibits in a subject one or more aspects of an autoimmune response selected from the group comprising production of type I interferon, production of autoantibodies, a mixed leukocyte reaction, a macrophage response, a natural killer reaction, and a lymphocyte activation. In a particular embodiment, production of type I interferon production is inhibited by a BST2 protein agent. In a preferred embodiment, production of type I interferon production is inhibited by a BST2 protein agent that is capable of binding an ILT7 receptor and stimulating an ILT7 receptor response in a cell.

In another embodiment, the invention provides a BST2 agent which can be recombinantly produced and which can be a full length BST2 protein, a portion of a BST2 protein corresponding to one or more extracellular domains of a BST2 protein, a fragment of a BST2 protein which retains ILT7 binding and stimulation capacity, or a BST2 fusion protein. In select embodiments, a BST2 protein agent may have an amino acid sequence of SEQ ID NO:1 or SEQ ID NO:2. In other embodiments, a BST2 protein agent can have an amino acid sequence which has at least 95% sequence identity with either of SEQ ID NO:1 or SEQ ID NO:2.

A particular aspect of the invention relates to a BST2 fusion protein which can comprise one or more extracellular domains of a BST2 protein linked to an immunoglobin Fc region, or a full-length BST2 protein linked to an immunoglobin Fc region.

The compositions and methods of treatment provided by the present invention may be useful in the treatment of autoimmune diseases including, but not limited to, systemic lupus erythematosus, cutaneous lupus erythematosus, Sjogren's syndrome, dermatomyositis, Goodpasture's syndrome, and psoriasis.

Given the diversity of autoimmune disease presentation, the invention provides several routes by which a pharmaceutical composition comprising a BST2 protein agent may be administered including, without limitation, intravenously, subcutaneously, intramuscularly, intrasynovially, mucosally, topically, by inhalation, by subconjunctival injection, and by intraglandular injection.

Certain embodiments of the present invention provide a pharmaceutical composition comprising a BST2 protein agent in an amount effective to decrease interferon production in a patient. A pharmaceutical composition may further comprises one or more pharmaceutically acceptable excipients, and may be formulated as appropriate to a variety of routes of administration.

In an aspect, a pharmaceutical composition of the present invention may comprise a BST2 protein agent which is selected from the group consisting of a full length BST2 protein, a portion of a BST2 protein corresponding to one or more extracellular domains of a BST2 protein, a fragment of a BST2 protein, and a BST2 fusion protein. In particular, the BST2 protein agent comprises an amino acid sequence having at least 95% sequence identity to SEQ ID NO:1 or SEQ ID NO:2.

Other objects, features and/or advantages of the present invention will become apparent from the following detailed description. It should be understood that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. Breast cancer cell line T47D expresses a potential ligand for ILT7. (FIG. 1A) Two human breast cancer cell lines were co-cultured with either ILT7+ NFAT-GFP reporter cells or parental NFAT-GFP reporter cells. The percentages of GFP-positive reporter cells were analyzed. (FIG. 1B) T47D cells were co-cultured with ILT7+ NFAT-GFP reporter cells in the presence of 1 gg/mL of control IgG1 or anti-ILT7 mAb. The percentages of GFPpositive reporter cells were plotted. (FIG. 1C) Breast cancer MDA-MB-468 cells were first cultured 5 days in the presence of medium, 5 ng/mL of TNFa, or 500 U/mL of IFNα, and then co-cultured with NFAT-GFP reporter cells. The percentages of GFP-positive reporter cells were analyzed.

FIGS. 2A-2D. Characterization of mAbs against a putative ILT7 ligand. (FIG. 1A) Culture supernatants from different hybridoma clones were included in the co-cultures of ILT7 reporter cells and T47D cells. The percentage of GFP-positive reporter cells were measured and analyzed. Two clones able to inhibit GFP induction are indicated (arrows). Anti-ILT7 mAb was included as a positive control. (FIG. 1B) MDA-MB-231 and T47D cells were stained with mAb 26F8 (red line) or an isotype-matched control mAb (shaded area) and analyzed by flow cytometry. A similar result obtained with mAb 28G4 is not shown. (FIG. 1C) T47D cells were co-cultured with ILT7 reporter cells in the presence of control IgG1, 26F8, or 28G4 mAbs at different concentrations. The percentages of GFP-positive reporter cells were plotted. (FIG. 1D) Surface biotinylated MDA-MB-231 and T47D were immunoprecipitated with control IgG1, 26F8, or 28G4. The precipitated proteins were analyzed by Western blotting with NeutrAvidin-HRP. Arrows indicate the specific protein bands obtained from T47D cells.

FIGS. 3A-3D. BST2 potently activates ILT7. (FIG. 3A) (left) HEK293 cells transiently transfected with mock control or BST2 cDNA were analyzed by Western blotting for BST2 protein expression. (right) Transfected cells were stained with 26F8 mAb and analyzed by flow cytometry. The staining profile with IgG isotype-matched control mAb is shown in the shaded area. Staining with 28G4 mAb produced identical results. (FIG. 3B) Plate-coated GST or BST2-GST were incubated with different concentrations of recombinant ILT7-Fc and then HRP-conjugated anti-human Fc. Shown is absorption of OD 450 nm from each sample after addition of Tetramethyl benzidine (TMB) substrate. (FIG. 3C) GST or BST2-GST protein was co-cultured with ILT7+ NFAT-GFP reporter cells. The percentages of GFP-positive reporter cells are shown. Neutralizing Abs alLT7 (5 pi·g/mL) or 26F8 (251.4 mL) or an IgG1 control antibody were included in the cultures, as indicated. (FIG. 3D) HEK293 cells transiently transfected with mock control or BST2 cDNA were co-cultured with ILT7+ NFAT-GFP reporter cells. The percentages of GFP-positive reporter cells are shown. Neutralizing Abs alLT7 (5 lag/mL), 26F8 (25 μg/mL), 28G4 (25 μg/mL), or control IgG1 were included in the cultures, as indicated.

FIGS. 4A-4D. BST2 activates primary pDCs and inhibits IFN and cytokine production by pDCs. (FIG. 4A) pDCs were incubated with anti-ILT7, recombinant Fc, or BST2-Fc proteins and analyzed for calcium influx. pDCs pre-treated with 5 1.1M of Syk inhibitor were also analyzed. (FIG. 4B) The amounts of secreted cytokines from pDCs cultured with plate-bound Fc or BST2-Fc are shown. The pDCs were activated overnight with either 0.2 μM of CpG 2216 or MO1 6 of Flu. Data from a representative donor is shown (n>8). (FIG. 4C) The levels of gene transcripts from pDCs cultured with purified Fc or BST2-Fc are shown. The relative expression of each gene was normalized with S18 and calculated against that of total PBMCs. (FIG. 4D) The amounts of secreted IFNα from Flu-challenged pDCs cultured with HEK293 with or without surface HA-tagged BST2 are shown. Data from a representative donor is shown (n>5).

FIGS. 5A-5B. Several breast cancer cells express ILT7-L. (FIG. 5A) Three breast cancer lines were co-cultured with ILT7- or ILT7+ reporter cells. The GFP-positive reporter cells were analyzed. (FIG. 5B) FACS staining of the breast cancer lines with mAb 26F8 generated against I LT7-L.

FIG. 6. BST2 does not affect costimulatory molecule expression by pDCs. pDCs cultured with plate-bound Fc or BST2-Fc and then activated with 0.2 pM of CpG 2216 for 48 hrs. Surface levels of CD80 and CD86 are shown. Also shown is the level of pDC marker CD123.

FIG. 7. IFN treatment induces strong BST2 expression by a variety of cells. HEK293, NHDF (dermal fibroblast), HUVEC (human umbilical vein endothelial cells), and HaCat (keratinocyte cell line) cells, cultured in the absence or presence of 500 units/mL of IFNα for 48 hr, were stained with mAb 26F8.

### DESCRIPTION OF ILLUSTRATIVE EMBODIMENTS

The present invention is based, in part, on the discovery by the inventors that the cell surface-associated glycoprotein, bone marrow stromal cell antigen 2 (BST2) can bind to the immunoglobin-like transcript 7 (ILT7) receptor expressed on plasmacytoid dendritic cells (pDCs), and intiate a signaling cascade which can result in the strong inhibition of interferon production by pDCs.

Designated natural type I interferon (IFN)-producing cells (IPCs), pDCs are capable of producing large quantities of IFN upon sensing viral and host nucleic acids through Toll-like receptor (TLR) 7 and TLR9 (Gilliet et al., 2008; Colonna et al., 2004. Uncontrolled pDC activation and IFN production are implicated in lymphopenia (Kamphuis et al., 2006; Lin et al., 1998) and autoimmune diseases (Lande et al., 2007; Blanco et al., 2001; Marshak-Rothstein and Rifkin, 2007, including, but not limited to, systemic lupus erythematosus (SLE) and psoriasis. The discovery of a mechanism that negatively controls TLR7/9-mediated IFN production in pDCs therefore provides a means of treating autoimmune diseases associated with IFN overproduction.

### I. Interferon and Interferon-Producing Cells

Interferons are a type of cytokine produced by a variety of human cells in response to various stimuli, including foreign nucleic acids, foreign cells (including tumour cells), bacteria, and viral antigens. Once interferon is released by an interferon-producing cell (IPC), it interacts with specific receptors, either on the same cell or on other cells, by inducing effector proteins, and a response to the stimuli results. IPCs can include, but are not limited to, plasmacytoid dendritic cells (pDCs), peripheral blood mononuclear leukocytes (PBLs), lymphocytes, macrophages, fibroblasts, and endothelial cells.

Interferons are classified as type I, II, or III according to the receptor(s) through which they signal. The term “interferon” can be used to refer to any or all of these interferon types. Type I interferons, in particular, are produced in large quantities during an immune response to viral and bacterial pathogens, and have been shown induce a number of immune cell behaviors as well as the expression of several proinflammatory chemokines such as CXCL9 and 10. Importantly, the overproduction and/or actions of type I interferons have been implicated in a variety of autoimmune processes. One of the largest and most diversified producers of type I interferons and as well as proinflammatory cytokines is a pDC.

In humans and other mammals, pDCs produce several different cytokines, circulate in the blood of adults and neonates (O'Doherty et al., 1994; Sorg et al., 1999) and can be located in lymphoid tissues (LNs, tonsils, spleen, thymus, bone marrow, and Peyer's patches) and certain peripheral tissues (fetal liver). Populations of pDCs also accumulate in inflammatory sites, e.g., lymphoid hyperplasia of the skin (Eckert and Schmid, 1989), systemic lupus erythematosus (SLE), psoriasis vulgaris (basal epidermis and papillary dermis, but not normal skin), contact dermatitis, and allergic mucosa (Wollenberg et al., 2002), and have been observed to infiltrate various primary and malignant tumors (Hartmann et al., 2003; Salio et al., 2003; Vermi et al., 2003; Zou et al., 2001). Recruitment into these sites suggests that pDCs may contribute to the ongoing inflammatory response through release of cytokines and chemokines and activation of lymphocytes (Yoneyama et al., 2002) or, alternatively, through the induction of tolerogenic responses (Zou et al., 2001).

Support for the role of pDCs as specialized immune cells in viral and bacterial, defense has come from observations that pDCs selectively express toll-like receptors 7 and 9 (TLR7 and TLR9, respectively), key endosomal sensors of microbial and “self” RNA or DNA, respectively (Kadowaki et al., 2001; Jarrossay et al., 2001; Hornung et al., 2002. Activation of TLR7 or TLR9 on pDCs by nucleic acids triggers discrete signal transduction, leading to rapid and robust secretion of type I interferon, inflammatory cytokines, and chemokines (Kawai and Akira, 2006; Honda and Taniguchi, 2006).

The interferon production in pDCs triggered by TLR 7 and/or TLR9 can be regulated by a number of signaling receptors called immunoreceptor tyrosine-based activation motif (ITAM) receptors, which are uniquely expressed on pDCs (Gilliet et al., 2008; Cao et al., 2007; Rock et al., 2007; Cao et al., 2006). One such receptor is ILT7, a member of the immunoglobulin (Ig)-like transcript family (ILT), also known as leukocyte Ig-like receptors (LILRs)) found in humans and primates (Brown et al., 2004).

The member proteins of the ILT family are expressed throughout the immune system, and represent a group of inhibitory receptors bearing immunoreceptor tyrosine-based inhibitory motifs (ITIM) as well as a few stimulatory receptors that signal through their association with adaptor molecules containing ITAM16. In particular, ILT7 (also known as LILRA4 and CD85g) contains four extracellular Ig-like domains and a positively charged residue within the transmembrane region, allowing ILT7 to form a receptor complex with a signaling adaptor protein, Fc epsilon RI gamma (FcεRIγ). Uniquely expressed by human pDCs, ILT7 was shown to suppress TLR7/9-induced IFN secretion by pDCs when crosslinked by an anti-ILT7 monoclonal antibody (Cho et al., 2008).

Several of the inhibitory ILTs regulate innate and adaptive immune responses through interaction with classical and non-classical major histocompatibility complex (MHC) class I ligands or viral-encoded MHC class 1-like proteins (Brown et al., 2004; Cho et al., 2008; Chapman et al., 1999). However, stimulatory ILTs, such as ILT7, have previously been uncharacterized with respect to non-MHC ligands. The current invention stems, in part, from the discovery of BST2 as a ligand which stimulates ILT7 to form a complex with FcεRIγ, thus initiating the signal which abrogates interferon type I production in pDCs.

### II. BST2 Protein

The bone marrow stromal antigen 2 (BST2) is a type II membrane glycoprotein which may be involved in pre-B cell growth (Goto et al., 1994; Ishikawa et al., 1995). BST2 is overexpressed in a number of cancers (Ohtomo et al., 1999), and has been shown to play a role in retroviral release from cells during HIV infection (Neil et al., 2008). BST2 is known by other names, including CD317, HM1.24, and tetherin, and its expression is reportedly upregulated by IFN in several immune cell types (Van damme et al., 2008).

As demonstrated in Example 1 herein, BST2 directly binds ILT7, initiates signaling by the ILT7/FcεRIγ receptor complex, and strongly inhibits production of type I interferon and proinflammatory cytokines by human pDCs stimulated with TLR7/9 ligands. Without wishing to be bound by any theory, the induction of BST2 expression by IFN and the inhibition of IFN production by BST2 may be an indication that the BST2-ILT7 interaction represents an important negative feedback loop modulating pDC's IFN responses. Inventors anticipate that such a mechanism which may be used by the human immune system to control innate immune responses and safeguard against autoimmunity.

The term “BST2 protein” includes BST2 (also referred to as CD317, HM1.24, and tetherin) from any species or source, and includes a full length BST2 protein as well as fragments or portions of the protein which are capable of binding ILT7 and inhibiting interferon production in an IPC. A BST2 protein of the current invention may be a protein comprising the amino acid sequence of SEQ ID: NO 1. A BST2 protein may be a mammalian BST2, or more particularly, a human BST2 or mouse BST2 protein. In other embodiments, a BST2 protein of the current invention may be a portion of the BST2 protein comprising one or more extracellular domains of BST2 or a portion of a BST2 protein having the amino acid sequence of SEQ ID: NO 2.

In some embodiments, the present invention provides a BST2 fusion protein. The fusion of particular moieties or domains to peptide or protein agents as a means of improving pharmacokinetic and pharmacodynamic properties is well known. Examples of such fusion domains include, but are not limited to, polyhistidine, Glu-Glu, glutathione S transferase (GST), thioredoxin, protein A, protein G, maltose binding protein (MBP), green fluorescent protein (GFP), various epitope tags such as FLAG, influenza virus hemagglutinin (HA), and c-myc, and cleavable fusion domains such as Factor Xa or Thrombin.

One commonly used fusion domain is an Fc region or a fragment of the immunoglobulin heavy chain constant region. A number of sequences within the constant region have been shown to enhance the serum half life of fused peptides and proteins, and several portions of the constant region are well established as activators of complement and as cellular effectors of the immune system. The Fc receptors are characterized in various cell types, as are the Fc regions from each of the immunoglobin classes with respect to the responses they elicit. An Fc region contemplated by the present invention may include any portion of a heavy chain constant region of an immunoglobin, in particular, an IgG or an IgE, which is capable of inducing calcium influx in pDCs, extending serum half-life and/or activating complement. In some aspects, an Fc region can be derived from a mammalian immunoglobin, in particular, a human immunoglobin.

Accordingly, certain embodiments of the present invention provide a BST2 protein which is prepared and administered as a soluble fusion protein, referred to herein as a “BST2 fusion protein”. In one aspect, the fusion protein may comprise one or more extracellular domains of BST2 linked to an immunoglobulin (Ig) Fc region. In another aspect, the BST2 fusion protein may comprise an immunoglobin Fc fragment linked to a fragment or portion of BST2 having an amino acid sequence at least 95% identical to SEQ ID NO:2. In yet other aspects, the BST2 fusion protein may contain a full length BST2 linked to an immunoglobulin (Ig) Fc region.

The term “BST2 protein agent” as used herein to refers to (a) a full length BST2 protein as disclosed herein, (b) a fragment or portion of a BST2 protein which retains ILT7 binding and inhibits interferon production by pDCs, (c) a protein comprising one or more extracellular domains of a BST2 protein which retains ILT7 binding and inhibits interferon production by pDCs, (d) a BST2 fusion protein comprising a full length BST2 protein linked to an immunoglobin Fc region, (e) a BST2 fusion protein comprising SEQ ID NO:2 linked to an immunoglobin Fc region, or (f) a BST2 fusion protein comprising one or more extracellular domains of a BST2 protein linked to an immunoglobin Fc region.

In some embodiments, a BST2 protein agent is a protein having the amino acid sequence of SEQ ID NO:1 or having at least 90%, at least 95%, at least 96%, at least 97%, at least 98%, or at least 99%, at least 99.2%, or more amino acid sequence identity with SEQ ID NO:1. In some embodiments, a BST2 protein agent is a protein having the amino acid sequence of SEQ ID NO: 2 or having at least 90%, at least 95%, at least 962%, at least 97%, at least 98%, or at least 99%, at least 99.2%, or more amino acid sequence identity with SEQ ID NO:2.

In other embodiments, a BST2 protein agent may be modified to be more therapeutically effective or suitable. For example, a BST2 protein agent may be converted into pharmaceutical salts by reaction with inorganic acids including hydrochloric acid, sulphuric acid, hydrobromic acid, phosphoric acid, or organic acids including formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and toluenesulphonic acids.

In a further embodiment of the instant invention, a BST2 protein agent may be modified to contain a fluorescent label or a complexing agent for radionuclides. The resulting labeled BST2 protein agent can be used to identify cells expressing ILT7 receptors, to quantify ILT7 receptor expression on a cell surface, and to identify, by a competitive assay known in the art, other ligands of the ILT7 receptor.

### III. Biological Equivalents of BST2

Preferred fragments or portions of the BST2 protein are those that are sufficient to bind an ILT7 protein specifically, thereby reducing interferon production in a cell. Determining whether a particular BST2 protein can bind ILT7 and reduce the production of interferon in a cell can be assessed using known in vitro immune assays including, but not limited to, inhibiting a mixed leucocyte reaction; inhibiting interferon production, inhibiting a cytotoxic T cell response; inhibiting interleukin-2 production; inhibiting an autoimmune response, inhibiting a Th1 cytokine profile; inducing IL-4 production; inducing TGF-β production; inducing IL-10 production; inducing a Th2 cytokine profile; inhibiting immunoglobulin production; and any other assay that would be known to one of skill in the art to be useful in detecting decreased interferon and/or immunosuppression.

A BST2 protein agent of the present invention may be modified to contain amino acid substitutions, insertions and/or deletions that do not alter their respective inhibition of IFN production or overall immunosuppressive properties. Such a biologically functional equivalent of BST2 could be a molecule having like or otherwise desirable characteristics, i.e. stimulation of ILT7 receptor and subsequent signaling blockade of IFN production. As a nonlimiting example, certain amino acids may be substituted for other amino acids in a BST2 protein structure without appreciable loss of interactive capacity, as demonstrated by unchanged ILT7 binding and sequelae. It is thus contemplated that a BST2 protein agent (or DNA encoding such an agent) which is modified in sequence and/or structure but which is unchanged in biological utility or activity remains within the scope of the present invention.

It is also well understood by the skilled artisan that, inherent in the definition of a biologically functional equivalent protein or protein fragment, is the concept that there is a limit to the number of changes that may be made within a defined portion of the molecule and still result in a molecule with an acceptable level of equivalent biological activity. Biologically functional equivalent proteins or protein fragments are thus defined herein as those proteins in which certain, not most or all, of the amino acids may be substituted. Of course, a plurality of distinct proteins or protein fragments with different substitutions may easily be made and used in accordance with the invention.

The skilled artisan is also aware that where certain residues are shown to be particularly important to the biological or structural properties of a protein or peptide, e.g., residues in active sites, such residues may not generally be exchanged. This is the case in the present invention, where any changes in the ILT7-binding region of BST2 that render a protein or fusion protein incapable of initiating ILT7-mediated suppression of IFN production would result in a loss of utility of the resulting protein or fusion protein for the present invention.

Amino acid substitutions, such as those which might be employed in modifying BST2 are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

The invention also contemplates isoforms of the proteins of the invention. An isoform contains the same number and kinds of amino acids as a protein of the invention, but the isoform has a different molecular structure. The isoforms contemplated by the present invention are those having the same properties as a protein of the invention as described herein.

In another embodiment of the instant invention, an ILT7 receptor ligand is provided. This ligand is sufficient to stimulate ILT7 and reduce the production of interferons by a cell, in particular, by a pDC. In one aspect, the ILT7 receptor ligand can be selected from the group comprising a full-length BST2 protein, a protein comprising a portion of BST2 corresponding to its extracellular domain, a BST2 fusion protein, a BST2 protein having the sequence of SEQ ID NO:1, and a BST2 protein having the sequence of SEQ ID NO:2. An ILT7 receptor ligand may be incorporated into a pharmaceutical composition and used to treat an autoimmune disease. In particular embodiments, an ILT7 ligand may be used to treat systemic lupus erythematosus, cutaneous lupus erythematosus, Sjogren's, syndrome, dermatomyositis, and psoriasis.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The amino acids described herein are preferred to be in the “L” isomeric form. However, residues in the “D” isomeric form can be substituted for any L-amino acid residue, as long as the desired functional properties set forth herein are retained by the protein. In keeping with standard protein nomenclature, J. Biol. Chem., 243:3552-59 (1969), abbreviations for amino acid residues are known in the art.

Nonstandard amino acids may be incorporated into proteins by chemical modification of existing amino acids or by de novo synthesis of a protein/peptide. A nonstandard amino acid refers to an amino acid that differs in chemical structure from the twenty standard amino acids encoded by the genetic code. Post-translational modification in vivo can also lead to the presence of a nonstandard or amino acid derivative in a protein. The N-terminal and C-terminal groups of a protein can also be modified, for example, by natural or artificial post-translational modification of a protein. Conservative substitutions are least likely to drastically alter the activity of a protein. A “conservative amino acid substitution” refers to replacement of amino acid with a chemically similar amino acid, i.e. replacing nonpolar amino acids with other nonpolar amino acids; substitution of polar amino acids with other polar amino acids, acidic residues with other acidic amino acids, etc.

In select embodiments, the present invention contemplates a chemical derivative of a BST2 protein agent. “Chemical derivative” refers to a protein having one or more residues chemically derivatized by reaction of a functional side group, and retaining biological activity and utility. Such derivatized proteins include, for example, those in which free amino groups have been derivatized to form specific salts or derivatized by alkylation and/or acylation, p-toluene sulfonyl groups, carbobenzoxy groups, t-butylocycarbonyl groups, chloroacetyl groups, formyl or acetyl groups among others. Free carboxyl groups may be derivatized to form organic or inorganic salts, methyl and ethyl esters or other types of esters or hydrazides and preferably amides (primary or secondary). Chemical derivatives may include those proteins which contain one or more naturally occurring amino acids derivatives of the twenty standard amino acids. For example, 4-hydroxyproline may be substituted for serine; and ornithine may be substituted for lysine.

### IV. Nucleic Acids

One of ordinary skill in art can easily appreciate that the amino acid changes described above may be effected by alteration of the encoding DNA; taking into consideration also that the genetic code is degenerate and that two or more codons may code for the same amino acid.

In accordance with the present invention there may be employed conventional molecular biology, microbiology, and recombinant DNA techniques, all within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins Eds. (1985)]; “Transcription and Translation” [B. D. Hames & S. J. Higgins Eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984). Other employed techniques may be peptide synthetic (Stewart and Young, 1984), analytical chemistry (Miller et al., 1996), structure activity relationship approaches (including in vivo and in vitro testing and structural analysis using NMR, CD, X-ray crystallography among others) (Gulyas et al., 1995).

As used herein, the term “cDNA” shall refer to the DNA copy of the mRNA transcript of a gene. As used herein, the term “derived amino acid sequence” shall mean the amino acid sequence determined by reading the triplet sequence of nucleotide bases in the cDNA.

As used herein the term “screening a library” shall refer to the process of using a labeled probe to check whether, under the appropriate conditions, there is a sequence complementary to the probe present in a particular DNA library. In addition, “screening a library” could be performed by PCR.

As used herein, the term “PCR” refers to the polymerase chain reaction that is the subject of U.S. Pat. Nos. 4,683,195 and 4,683,202 to Mullis, as well as other improvements now known in the art.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence, which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence. Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included near the coding sequence. This sequence encodes a signal peptide, N-terminal to the protein, which communicates to the host cell to direct the protein to the cell surface or secrete the polypeptide into the media, and this signal peptide is clipped off by the host cell before the protein leaves the cell. Signal sequences can be found associated with a variety of proteins native to prokaryotes and eukaryotes.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementary with the sequence or hybridize therewith and thereby form the template for the synthesis of the extension product.

As used herein, the terms “restriction endonucleases” and “restriction enzymes” refer to enzymes, each of which cut double-stranded. DNA at or near a specific nucleotide sequence.

A “clone” is a population of cells derived from a single cell or ancestor by mitosis.

A “cell line” is a clone of a primary cell that is capable of stable growth in vitro for many generations.

Two DNA sequences are “substantially homologous” when at least about 75% (preferably at least about 80%, and most preferably at least about 90% or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, for example, Maniatis et al., supra; DNA Cloning, Vols. I & II. supra; Nucleic Acid Hybridization, supra.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. In another example, the coding sequence is a construct where the coding sequence itself is not found in nature (e.g., a cDNA where the genomic coding sequence contains introns or synthetic sequences having codons different than the native gene). Allelic variations or naturally occurring mutational events do not give rise to a heterologous region of DNA as defined herein.

As used herein, the term “host” is meant to include not only prokaryotes but also eukaryotes such as yeast, plant and animal cells. A recombinant DNA molecule or gene that encodes a protein of the present invention can be used to transform a host using any of the techniques commonly known to those of ordinary skill in the art. Prokaryotic hosts may include E. coli, S. tymphimurium, Serratia marcescens and Bacillus subtilis. Eukaryotic hosts include yeasts such as Pichia pastoris, mammalian cells and insect cells. In general, expression vectors containing promoter sequences that facilitate the efficient transcription of the inserted DNA fragment are used in connection with the host. The expression vector typically contains an origin of replication, promoter(s), terminator(s), as well as specific genes that are capable of providing phenotypic selection in transformed cells. The transformed hosts can be fermented and cultured according to means known in the art to achieve optimal cell growth.

Methods well known to those skilled in the art can be used to construct expression vectors containing appropriate transcriptional and translational control signals. See for example, the techniques described in Sambrook et al. (1989) A gene and its transcription control sequences are defined as being “operably linked” if the transcription control sequences effectively control the transcription of the gene. A BST2 protein gene or fusion protein cDNA and its control sequences may be comprised in a vector. Vectors of the invention include, but are not limited to, plasmid vectors and viral vectors.

Analogs of a protein of the invention may be prepared by introducing mutations in the nucleotide sequence encoding the protein. Mutations in nucleotide sequences constructed for expression of analogs of a protein of the invention must preserve the reading frame of the coding sequences. Furthermore, the mutations will preferably not create complementary regions that could hybridize to produce secondary mRNA structures, such as loops or hairpins, which could adversely affect translation of the receptor mRNA.

Mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site specific mutagenesis procedures may be employed to provide an altered gene having particular codons altered according to the substitution, deletion, or insertion required. Deletion or truncation of a protein of the invention may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al. (1989).

The term “isolated” refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded.

Preferably, the isolated nucleic acid molecule which is useful in the present invention can comprise (a) a nucleic acid encoding a full-length BST2 having the amino acid sequence of SEQ. ID. NO.:1, (b) a nucleic acid sequences complementary to (a); (c) a nucleic acid encoding a BST2 protein having the amino acid sequence of SEQ. ID. NO.:2, (d) a nucleic acid encoding one or more extracellular domains of a BST2 protein linked to an immunoglobin Fc region, or (e) a nucleic acid molecule differing from any of the nucleic acids of (a) or (c) in codon sequences due to the degeneracy of the genetic code.

It will be appreciated that the invention includes nucleic acid molecules encoding truncations of a BST2 protein agent of the invention, and analogs and homologs of a protein of the invention and truncations thereof, as described below. It will further be appreciated that variant forms of the nucleic acid molecules of the invention which arise by alternative splicing of an mRNA corresponding to a cDNA of the invention are encompassed by the invention.

An isolated nucleic acid molecule of the invention which is DNA can also be isolated by selectively amplifying a nucleic acid encoding a novel protein of the invention using the polymerase chain reaction (PCR) methods and cDNA or genomic DNA. It is possible to design synthetic oligonucleotide primers from the nucleic acid molecule encoding a BST2 protein agent for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis.

It will be appreciated that cDNA may be prepared from mRNA, by isolating total cellular mRNA by a variety of techniques, for example, by using the guanidinium-thiocyanate extraction procedure of Chirgwin et al. (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a novel protein of the invention into an appropriate vector which allows for transcription of the cDNA to produce an RNA molecule which encodes a BST2 protein agent of the invention. For example, a cDNA can be cloned downstream of a bacteriophage promoter, (e.g. a T7 promoter) in a vector, cDNA can be transcribed in vitro with T7 polymerase, and the resultant RNA can be isolated by standard techniques.

A nucleic acid molecule of the invention may also be chemically synthesized using standard techniques. Various methods of chemically synthesizing polydeoxy-nucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

The sequence of a nucleic acid molecule of the invention may be inverted relative to its normal presentation for transcription to produce an antisense nucleic acid molecule. Preferably, an antisense sequence is constructed by inverting a region preceding the initiation codon or an unconserved region. In particular, the nucleic acid sequences contained in the nucleic acid molecules of the invention or a fragment thereof, preferably a nucleic acid sequence encoding a protein having a sequence disclosed herein, may be inverted relative to its normal presentation for transcription to produce antisense nucleic acid molecules.

### V. Methods of Producing a BST2 Protein Agent

A BST2 protein agent may be obtained from known sources or prepared using recombinant techniques known in the art. In the case of a BST2 protein, this will generally involve the construction of a DNA sequence encoding a full length BST2 or fragment or portion thereof in an expression construct or vector, and producing the BST2 protein in the appropriate expression system. For a BST2 fusion protein, a DNA sequence encoding a full length BST2 or one or more extracellular domains of BST2 is linked to a DNA sequence encoding an immunoglobin Fc region and the resulting complex is expressed in an appropriate expression system where the BST2-Fc fusion protein is produced. By way of non-limiting example, a representative method of producing a BST2 protein agent may include purification or isolation of the BST2 agent from stably or transiently transformed bacterial, yeast, insect or mammalian cell cultures following transfection of the cultures with a nucleic acid encoding the BST2 protein agent.

Accordingly, a nucleic acid molecule encoding a BST2 protein agent of the present invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, means that the expression vectors contain a nucleic acid molecule of the invention and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The invention therefore contemplates a recombinant expression vector of the invention containing a nucleic acid encoding a BST2 protein agent of the invention, or a fragment thereof, and the necessary regulatory sequences for the transcription and translation of the inserted protein-sequence. Such expression vectors may be useful in the above-described therapies using a nucleic acid sequence encoding a BST2 protein agent. Suitable regulatory sequences may be derived from a variety of sources, including bacterial, fungal, or viral genes (For example, see the regulatory sequences described in Goeddel (1990).

Selection of appropriate regulatory sequences is dependent on the host cell chosen, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native protein and/or its flanking regions.

The invention further provides a recombinant expression vector comprising a DNA nucleic acid molecule of the invention cloned into the expression vector in an antisense orientation. That is, the DNA molecule is operatively linked to a regulatory sequence in a manner which allows for expression, by transcription of the DNA molecule, of an RNA molecule which is antisense to a nucleotide sequence encoding a BST2 protein agent disclosed herein. Regulatory sequences operatively linked to the antisense nucleic acid can be chosen which direct the continuous expression of the antisense RNA molecule.

A recombinant expression vector of the present invention may also contain a selectable marker gene which facilitates the selection of host cells transformed or transfected with a recombinant BST2 protein agent. Examples of selectable marker genes are genes encoding a protein such as G418 and hygromycin which confer resistance to certain drugs, β-galactosidase, chloramphenicol acetyltransferase, or firefly luciferase. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of a recombinant expression vector, and in particular, to determine the effect of a mutation on expression and phenotype. It will be appreciated that selectable markers can be introduced on a separate vector from the nucleic acid of interest.

In other embodiments, a recombinant expression vector may contain one or more genes which encode a fusion moiety which provides increased expression of the recombinant protein; increased solubility of the recombinant protein; and aid in the purification of a target recombinant protein by acting as a ligand in affinity purification. For example, a proteolytic cleavage site may be added to the target recombinant protein to allow separation of the recombinant protein from the fusion moiety subsequent to purification of the fusion protein.

Recombinant expression vectors can be introduced into host cells to produce a transformant host cell. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (1989), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells or mammalian cells. Other suitable host cells can be found in Goeddel (1991).

A BST2 protein agent of the invention may also be prepared by chemical synthesis using techniques well known in the chemistry of proteins such as solid phase synthesis (Merrifield, 1964) or synthesis in homogenous solution (Houbenweyl, 1987).

### VI. Autoimmune Diseases and Methods of Treatment Thereof

In one aspect, the present invention provides a method of treating an autoimmune disease wherein a subject is identified as having or as suspected of having an autoimmune disease, and a pharmaceutical composition comprising a therapeutically effective amount of a BST2 protein agent or a nucleic acid sequence encoding a BST2 protein agent is administered to the subject. In particular embodiments, the pharmaceutical composition used to treat an autoimmune disease according to the present invention comprises a BST2 fusion protein or a nucleic acid encoding a BST2 fusion protein. In other embodiments, the pharmaceutical composition additionally comprises one or more pharmaceutically acceptable excipients.

Administration of an “effective amount” of a BST2 protein agent or a nucleic acid encoding a BST2 protein agent is defined as an amount effective, at dosages and for periods of time necessary to achieve the desired result. The effective amount of a BST2 protein agent or a nucleic acid encoding a BST2 protein agent may vary according to factors such as the disease state, age, sex, and weight of the subject. Using methods well known in the clincal arts, dosage regima may be adjusted to provide the optimum therapeutic response. For example, several divided doses may be administered daily or the dose may be proportionally reduced as indicated by the exigencies of the therapeutic situation.

The terms “subject” and “patient” are used interchangeably and can include all mammals, especially humans.

Autoimmune diseases that may be treated according to the present invention include, without limitation, arthritis, type I insulin-dependent diabetes mellitus, adult respiratory distress syndrome, inflammatory bowel disease, dermatitis, thrombotic thrombocytopenic purpura, Sjogren's syndrome, encephalitis, uveitis, leukocyte adhesion deficiency, rheumatoid arthritis, rheumatic fever, Reiter's syndrome, psoriatic arthritis, progressive systemic scleroderma, primary biliary cirrhosis, pemphigus, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune thrombocytopenia, autoimmune haemolytic anemia, Hashimoto's thyroiditis, Goodpasture's syndrome, Graves disease, habitual spontaneous abortions, Reynard's syndrome, glomerulonephritis, dermatomyositis, chronic active hepatitis, celiac disease, psoriasis, tissue specific autoimmunity, autoimmune polyendocrinopathy syndrome, degenerative autoimmunity delayed hypersensitivities, autoimmune complications of AIDS, atrophic gastritis, ankylosing spondylitis and Addison's disease.

For most of these diseases, symptoms vary so dramatically among patient groups, and even occasionally, along the course of the disease in a single patient, that symptoms are used mostly as a basis for suspecting an autoimmune etiology or for ruling out other causes that are not autoimmune in nature. A medical doctor of ordinary skill is familiar with symptoms associated with individual forms of autoimmune disease which would identify a subject as having or as suspected of having a particular autoimmune disease. By way of nonlimiting example, selected autoimmune diseases can be associated with symptoms described in Table I.

Due to the variable symptoms of autoimmune disease, the definitive and most reliable step in the diagnosis for many autoimmune diseases, is a blood test designed to identify the presence of specific antibodies (i.e., autoantibodies) associated with the suspected autoimmune response. Because the pathophysiology of an autoimmune disease is, in part, the result of specific, sustained, and recurring autoimmune responses, the antibodies generated during such responses are usually reliable markers for these diseases. Appropriate antibody tests have been identified in the art and are in regular clinical use for most of the currently known forms of autoimmune disease. For example, the anti-nuclear antibody (ANA) test, is performed in all suspected cases of systemic autoimmune disease including, without limitation, systemic lupus erythematosus, Sjogren's syndrome, scleroderma, rheumatoid arthritis, autoimmune vasculitis.

TABLE I
Some Representative Autoimmune Diseases

| Disease Name | Symptoms |
|---|---|
| Systemic | fever, chills, fatigue, weight loss, skin rashes, |
| lupus erythematosus | patchy hair loss, nasal and oral sores, irregular |
| | menstrual periods |
| Rheumatoid arthritis | fever, loss of appetite, weight loss, symmetrical |
| | joint pain, swelling and stiffness |
| Goodpasture's | fatigue, paleness, bleeding in the lungs and rapid |
| syndrome | destruction of kidney tissue |
| Grave's disease | enlarged thyroid gland, weight loss, sweating, |
| | irregular heart beat, nervousness, heat sensitivity |
| Hashimoto's | thyroid dysfunction |
| thyroiditis | |
| Sjögren's syndrome | excessive dry eye and dry mouth |
| Type I diabetes | fatigue and hyperglycemia |
| mellitus | |
| Psoriasis | Scaly red or silvery skin lesions, skin pain and |
| | inflammation, nail thickening and splitting, arthritis |
| Myasthenia gravis | muscle weakness, swallowing and breathing |
| | difficulties, paralysis |
| Scleroderma | joint pain, swelling, and stiffness, skin tightness, |
| | shininess, weight loss, loss of appetite, intestinal |
| | disturbances |

Accordingly, the identification of a subject having or suspected of having an autoimmune disease can refer to a combination of symptoms and a diagnostic result whereby the presence and/or level in the subject of one or more disease-specific antibodies or autoantibodies is indicative of the autoimmune disease. By way of a nonlimiting example, a patient suspected of having an organ-specific autoimmune disease of the kidney based on symptoms would be identified as having the disease if a sample from the patient revealed the presence of kidney-specific autoantibodies.

Lupus erythematosus is a group of chronic autoimmune diseases, the two most common of which are systemic lupus erythematosus (SLE) and cutaneous or discoid lupus erythematosus (CLE). Despite the greater worldwide prevalence of CLE, the word “lupus” is most often used to refer only to SLE. One reason may be that SLE can affect multiple organ systems, including the heart, skin, joints, kidneys, and nervous system, and can be fatal. Though fatalities are more rare because of advances in detection, SLE almost always involves chronic inflammation and tissue damage and is still representative among autoimmune diseases as a debilitating and painful condition.

The American College of Rheumatology (ACR) has established criteria for the diagnosis or identification of a patient with SLE as including a positive antinuclear antibody test, and three symptoms selected from the group comprising serositis, oral ulcers, arthritis, photosensitivity, hematological changes, proteinuria, immunological changes, neurological signs (i.e., seizures, psychosis), malar rash, and discoid rash. Using these criteria, a medical doctor of ordinary skill is be able to identify a subject as having or as suspected of having SLE.

Recently, the study of systemic lupus erythematosus (SLE) has revealed a central role for type I interferons (IFNs) in SLE pathogenesis. Type I IFNs induce the unabated activation of pDCs, which select and activate autoreactive T cells rather than deleting them, thus failing to induce immune tolerance and encouraging autoimmunity. IFN also directly affects T cells and B cells. Furthermore, the activation of Toll-like receptors on pDCs provides an amplification loop for IFN production and B-cell activation in SLE.

Accordingly, the present invention provides a method of treating SLE comprising identifying a patient having SLE or suspected of having SLE and administering a therapeutically effective amount of a pharmaceutical composition containing a BST2 protein agent to the patient. In particular embodiments, a pharmaceutical composition for treating SLE can comprise a BST2 protein agent and one or more pharmaceutically acceptable excipients. In other embodiments, a pharmaceutical composition for treating SLE may be administered topically, intralesionally, intravenously, or mucosally.

Cutaneous lupus erythematosus (CLE) is one of the most common dermatological autoimmune disorders worldwide. Diagnosis of CLE is similar to that for SLE, except that mostly skin symptoms are prevalent in CLE. A medical doctor of ordinary skill in the art will be able to identify a subject as having or as suspected of having CLE using one or more of the following diagnostic criterion: oral ulcers, positive specific antinuclear antibody tests, a malar rash, or a discoid rash. As with SLE, several studies have recently provided evidence for a pathogenic role of type I IFNs in CLE, and identified pDCs as the main source of excess IFN in CLE skin lesions.

Accordingly, a method is provided herein for the treatment of CLE comprising identifying a patient having CLE or suspected of having CLE and administering a therapeutically effective amount of a pharmaceutical composition containing a BST2 protein agent to the patient. In particular embodiments, a pharmaceutical composition for treating CLE can comprise a BST2 protein agent and one or more pharmaceutically acceptable excipients. In other embodiments, a pharmaceutical composition for treating CLE may be administered topically, intralesionally, or mucosally.

One of the most common and perhaps the oldest known of the autoimmune diseases, psoriasis remains surprisingly misunderstood and difficult to treat. Psoriasis affects the skin and joints, causing red scaly patches (called plaques) to appear on the skin denoting areas of inflammation and excessive skin production. Skin rapidly accumulates at these sites and takes on a characteristic silvery-white appearance.

Psoriasis is chronic and recurring and can vary in severity from minor localised patches to complete body coverage. Fingernails and toenails are frequently affected (psoriatic nail dystrophy), and in 10-15% of patients, psoriasis can also cause inflammation of the joints, which is known as psoriatic arthritis. On a molecular level, psoriasis has been linked with uncontrolled innate immunity, involving recruitment of lymphocytes, natural killer cells, and dendritic cells to psoriatic lesions as well as overproduction of type I interferons.

Accordingly, the present invention provides a method for the treatment of psoriasis, including psoriatic arthritis, in a subject. This method comprises the identification of a subject as having or suspected of having psoriasis and the administration of a therapeutically effective amount of a pharmaceutical composition containing a BST2 protein agent. In particular embodiments, a pharmaceutical composition for treating psoriasis can comprise a BST2 protein agent and one or more pharmaceutically acceptable excipients. In other embodiments, a pharmaceutical composition for treating psoriasis may be administered topically, intralesionally, or intrasynovially.

In particular embodiments, the present invention provides a method of treating Sjogren's syndrome, which is the second most common form of autoimmune rheumatic disease. It is a disorder in which immune cells attack and destroy the exocrine glands that produce tears and saliva. It overwhelmingly affects women and most often occurs after the age of 40. It is statistically associated with other autoimmune diseases such as rheumatoid arthritis.

Accordingly, a method of treating Sjogren's syndrome may comprise the identification of a subject having or suspected of having Sjogren's syndrome and the administration of a therapeutically effective amount of a pharmaceutical composition containing a BST2 protein agent. In particular embodiments, a pharmaceutical composition for treating Sjogren's syndrome can comprise a BST2 protein agent and one or more pharmaceutically acceptable excipients. In other embodiments, a pharmaceutical composition for treating Sjogren's syndrome may be administered topically, orally, intravenously, intraocularly, mucosally, or by subconjunctival or intraglandular injection.

In particular embodiments, the present invention provides a method of treating dermatomyositis, which is a connective-tissue disease characterized by inflammation of muscle and skin tissue. It is very commonly observed to overlap or be combined with other autoimmune diseases, and is often observed in cancer patients. In fact, clinical observation of a dermatomyositic rash in a patient with symmetric, proximal muscle weakness is generally followed up with an investigation of neoplastic disease.

Accordingly, a method of treating dermatomyositis may comprise the identification of a subject having or suspected of having dermatomyositis and the administration of a therapeutically effective amount of a pharmaceutical composition containing a BST2 protein agent. In particular embodiments, a pharmaceutical composition for treating dermatomyositis can comprise a BST2 protein agent and one or more pharmaceutically acceptable excipients. In other embodiments, a pharmaceutical composition for treating dermatomyositis may be administered topically, mucosally, intravenously, intrasynovially, intramuscularly, or by subconjunctival or intraglandular injection.

In particular embodiments, the present invention provides a method of treating Goodpasture's syndrome, which is one of the rarest forms of autoimmune disease and can result in fatal incidences of renal failure or pulmonary hemorrhage. Goodpasture's syndrome is generally diagnosed in a patient by the presence of autoantibodies to collagen-associated antigens which are specific to the alveolar and glomerular basement membranes. The critically important role of IL-2 and type I interferons in Goodpasture's pathology has recently been demonstrated (Queluz et al., 1990).

Accordingly, a method of treating Goodpasture's syndrome may comprise the identification of a subject having or suspected of having Goodpasture's syndrome and the administration of a therapeutically effective amount of a pharmaceutical composition containing a BST2 protein agent. In particular embodiments, a pharmaceutical composition for treating Goodpasture's syndrome can comprise a BST2 protein agent and one or more pharmaceutically acceptable excipients. In other embodiments, a pharmaceutical composition for treating Goodpasture's syndrome may be administered parenterally, mucosally, intravenously, or by inhalation.

Each of the terms “administering a therapeutically effective amount of a pharmaceutical composition comprising a BST2 protein agent” and “administering a therapeutically effective amount of a pharmaceutical composition containing a BST2 protein agent” includes both the administration of the BST2 protein agent as well as the administration of a nucleic acid sequence encoding a BST protein agent. In the latter, the BST2 protein agent is produced in vivo in the subject.

### VII. Interferon Production and ILT7 Dynamics

The treatment methods of the present invention have stemmed, in part, from the observations by the Applicant that BST2 directly binds ILT7, initiates signaling by the ILT7/FcεRIγ receptor complex, and strongly inhibits production of type I interferon and proinflammatory cytokines by human pDCs. Because BST2 expression is induced by IFN and other proinflammatory cytokines in a variety of cell types, the BST2-ILT7 interaction may also represent a useful tool in studying the modulation of IFN responses involving pDCs and other interferon producing cells. Therefore, contemplated in select embodiments is the exploitation of the unique BST2-ILT7 interaction in methods of reducing the production of interferon in a cell or a subject, identifying and characterizing other ILT7 ligands, and identifying ILT7 expression on a population of cells.

### A. Inhibiting Interferon Production in a Cell

Certain embodiments of the present invention pertain to methods of inhibiting interferon production in a cell. These methods involve contacting a cell with a BST2 protein agent, and measuring the interferon produced by the cell to determine if the amount of the interferon produced by the cell is decreased.

In some aspects, the invention provides for a method of inhibiting interferon production in a plasmacytoid dendritic cell (pDC) population which features a first pDC population contacted with a BST2 protein agent in a suitable carrier, a second pDC population contacted with said suitable carrier in the absence of the BST2 protein agent, a measurement of interferon by each of the first and second populations, and a comparison of the interferon produced by the first pDC population with the interferon produced by the second pDC population. In this method, a lesser amount of interferon produced by the first pDC population relative to the second pDC population is an indication that the production of interferon in a pDC population has been inhibited.

In other aspects, the invention provides for a method of inhibiting interferon production in a plasmacytoid dendritic cell (pDC) population which features a first measurement of interferon produced by the pDC population followed by an exposure of the population to a BST2 protein agent in a suitable carrier, and a second measurement of interferon produced by the pDC population, and a comparison of the interferon produced by the first pDC population with the interferon produced by the second pDC population. In this method, a lesser amount of interferon produced by the first pDC population relative to the second pDC population is an indication that the production of interferon in a pDC population has been inhibited.

A method of inhibiting the production of interferon in a cell can include inhibition of interferon production in any cell types which are known to produce interferon (IPCs) and may also include cell types which have yet to be characterized with respect to interferon production. Cell types which have been shown to produce interferons in humans and in which production of interferon may be inhibited include, but are not limited to, plasmacytoid dendritic cells, peripheral blood mononuclear leukocytes (PBLs), lymphocytes, macrophages, fibroblasts, and endothelial cells. In particular, type I interferon production can be inhibited in a plasmacytoid dendritic cell.

For the purposes of inhibiting interferon production in a cell, a suitable carrier in which to provide a BST2 protein agent to the cell can be any suitable carrier used for cellular treatment procedures in the art. The carrier can be, for example, a sterile aqueous-based solution, and can comprise a BST2 protein agent as well as one or more agents selected from agents regularly used in the art which are compatible with cell culture procedures. The production of interferon by a cell may likewise be assessed using any appropriate methods known in the art. By way of non-limiting example, the amount of type I IFN can be examined in a sample using a conventional gene reporter assay, an ELISA assay, an immune cell response assay, or an antiviral activity assay.

### B. Reducing Interferon Production in a Subject

In select embodiments, the invention provides a method of reducing the production of interferon in a subject. This method can comprise steps of (1) obtaining a first sample from a subject, (2) administering to the subject an ILT7 receptor ligand, (3) obtaining a second sample from a subject, (4) measuring the interferon in each of the first sample and the second sample, and (5) comparing the amount of interferon in the first sample with the amount of interferon in the second sample. Using this method, an assessment that the production of interferon in a patient has been reduced can be made if a lesser amount of interferon is found in the second sample relative to the first sample. In preferred embodiments, the ILT7 receptor ligand used in this method is a full length BST2 protein, a portion of a BST2 protein corresponding to one or more extracellular domains of a BST2 protein, a fragment of a BST2 protein that is capable of bind and stimulating an ILT7 receptor, or a BST2 fusion protein.

A method for reducing interferon production in a subject may be of particular use in the treatment of diseases or medical conditions which involve an overproduction of or an aberrant response to interferons including, but not limited to, chronic or acute inflammatory diseases, autoimmune diseases, infectious diseases, and proliferative diseases and disorders.

As with the measurement of interferon produced by a cell, the production of interferon in a subject can be assessed using any clinical methods known in the art. By way of non-limiting example, the amount of type I IFN can be examined in a blood or tissue sample from a subject using a conventional gene reporter assay, an ELISA assay, an immune cell response assay, or an antiviral activity assay. Particular clinical parameters for accuracy, variability, and the use of control samples in the design of such methods is well understood within the clinical laboratory art.

### C. Identifying an ILT7 Ligand

Contemplated in the present invention is a method of identifying and characterizing an ILT7 ligand. In an aspect, this method can include steps of (1) obtaining plasmacytoid dendritic cells expressing ILT7, (2) contacting the plasmacytoid dendritic cells with a detectably labeled BST2 protein agent alone or together with a sample containing a potential ILT7 ligand; and (3) determining the production of interferon by the plasmacytoid dendritic cells. In this case, a reduction in detectable binding of the labeled BST2 protein agent when the sample is present identifies an ILT7 ligand in the sample.

A method of identifying an ILT7 ligand disclosed herein may also be used to assess the strength or dynamics of BST2 binding to ILT7. The method of performing such an assessment would employ a particular assay system developed and utilized in the art known as a receptor assay or a competitive receptor assay. In a receptor assay, the material to be assayed is appropriately labeled and then a cell population bearing a receptor of interest are contacted with a quantity of both the labeled and non-labeled material after which binding studies are conducted to determine the extent to which the labeled material binds to the cell receptors. In this way, differences in affinity between materials can be compared.

The labels envisioned for use with methods disclosed herein can include, without limitation, radioactive elements, enzymes, chemicals that fluoresce when exposed to ultraviolet light, and others. A number of fluorescent materials are known and can be utilized as labels. These include, for example, fluorescein, rhodamine, auramine, Texas Red, AMCA blue and Lucifer Yellow.

### B. Identifying ILT7 Expression on a Cell

Similar to methods of identifying ILT7 ligands, a method of identifying ILT7 expression on a cell also features a receptor assay. In identifying expression of an ILT7 receptor using its expression on a cell surface however, the cells themselves may be analyzed by, for instance, fluorescence-activated cell sorting (FACS) or microscopy techniques. This method can comprise the steps of (1) contacting a quantified population of cells upon which the expression of ILT7 is unknown with a detectably labeled BST2 protein agent; and (2) measuring the amount of label associated with the cells or material derived therefrom. In this case, a detectable binding of the labeled BST2 protein agent to the cells identifies an ILT7 receptor is expressed on the cells. The same analysis can be used to quantify expression of an ILT7 receptor by adding the steps of measuring at least three different samples comprising known amounts of the labeled BST2 protein agent and comparing the known values with the measurement of label associated with the quantified population of cells. Since the number of cells is known and the label can be quantified by comparison with known amounts of the labeled BST2 protein agent, the amount of labeled BST2 protein agent, and thus, the level of ILT7 receptor on the surface of the cells, can be determined.

As with identification of ligands, methods of identifying and/or quantifying ILT7 receptors can employ a variety of appropriate labels including, without limitation, radioactive elements, enzymes, chemicals that fluoresce when exposed to ultraviolet light, and fluorescent labels such as fluorescein, rhodamine, auramine, Texas Red, AMCA blue and Lucifer Yellow.

### VIII. Pharmaceutical Compositions

The present invention provides one or more pharmaceutical compositions comprising one or more pharmaceutically acceptable excipients and a BST2 protein agent or a nucleic acid encoding a BST2 protein agent for use in treating an autoimmune disease or reducing interferon production. In an aspect, the pharmaceutical composition can comprise a BST2 protein agent selected from the group comprising (a) a BST2 protein as disclosed herein, (b) a fragment or portion of a BST2 protein which retains ILT7 binding and inhibits interferon production by pDCs, (c) a protein comprising one or more extracellular domains of a BST2 protein which retains ILT7 binding and inhibits interferon production by pDCs, (d) a BST2 fusion protein comprising a full length BST2 protein linked to an immunoglobin Fc region, (e) a BST2 fusion protein comprising a BST2 protein of SEQ ID NO:2 linked to an immunoglobin Fc region, and (f) a BST2 fusion protein comprising one or more extracellular domains of a BST2 protein linked to an immunoglobin Fc region. In particular, the BST2 protein agent comprises an amino acid sequence having at least 95% sequence identity to SEQ ID NO:1 or SEQ ID NO:2.

In certain embodiments of the present invention, a pharmaceutical composition comprises a BST2 protein agent in an amount effective to decrease interferon production in a patient. Such a composition may be formulated as appropriate to a variety of routes of administration.

In particular embodiments, a pharmaceutical composition can comprise one or more pharmaceutically acceptable excipients and an ILT7 receptor ligand selected from the group comprising a full length BST2 protein, a portion of a BST2 protein corresponding to one or more extracellular domains of a BST2 protein, a fragment of a BST2 protein, and a BST2 fusion protein.

In some embodiments, a pharmaceutical composition can include, albeit not exclusively, a BST2 protein agent in association with one or more pharmaceutically acceptable vehicles or diluents, and contained in buffered solutions with a suitable pH and isoosmotic with the physiological fluids. The pharmaceutical compositions may additionally contain other agents such as immunosuppressive drugs or antibodies to enhance immune tolerance. The term “active ingredient” is used herein to denote any of a BST2 protein agent, a nucleic acid encoding a BST2 protein agent, or an ILT7 receptor ligand.

The nucleic acid molecules of the invention encoding a BST2 protein agent may be used in gene therapy to promote immune tolerance. Recombinant molecules comprising a nucleic acid sequence encoding a BST2 protein agent may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes. Recombinant molecules may also be delivered in the form of an aerosol or by lavage. The nucleic acid molecules of the invention may also be applied extracellularly such as by direct injection into cells.

A pharmaceutical composition used in accordance with a method of the invention can be an aerosolized powder or liquid, a liquid, a solid or a semisolid and can be formulated in, for example, pills, tablets, creams, ointments, inhalants, gelatin capsules, capsules, suppositories, soft gelatin capsules, gels, membranes, tubelets, solutions or suspensions.

A pharmaceutical composition of the present invention may comprise different types of carriers depending on whether it is to be administered in solid, liquid or aerosol form, and whether it need to be sterile for such routes of administration as injection. A pharmaceutical composition of the invention can be intended for administration to humans or other animals. Dosages to be administered depend on individual needs, on the desired effect and on the chosen route of administration.

In accordance with the present invention, a pharmaceutical composition containing a BST2 protein agent can be administered intravenously, intradermally, intraarterially, intraperitoneally, intralesionally, intracranially, intraarticularly, intraprostaticaly, intrapleurally, intrasynovially, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularly, orally, topically, by inhalation, infusion, continuous infusion, localized perfusion, via a catheter, via a lavage, in lipid compositions (e.g., liposomes), or by other method or any combination of the forgoing as would be known to one of ordinary skill in the art.

A pharmaceutical composition can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (2005).

In an aspect, a pharmaceutical composition of the present invention can comprise a therapeutically effective amount of a BST2 protein agent or nucleic acid encoding a BST2 protein agent. The phrase “pharmaceutical or pharmacologically acceptable” or “therapeutically effective” refers to molecular entities and compositions that do not produce an adverse, allergic or other untoward reaction when administered to a subject, such as, for example, a human, as appropriate. The phrase “therapeutically effective amount” refers to an amount of a composition required to achieve a desired medical result, in particular, to achieve the treatment of an autoimmune disease. The preparation of therapeutically effective compositions will be known to those of skill in the art in light of the present disclosure, as exemplified by Remington's Pharmaceutical Sciences (2005), incorporated herein by reference. Moreover, for animal (e.g., human) administration, it will be understood that preparations should meet sterility, pyrogenicity, general safety and purity standards as required by FDA Office of Biological Standards.

As used herein, “a composition comprising a therapeutically effective amount” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with a BST2 protein agent, its use in the present compositions is contemplated.

The actual required amount of a composition of the present invention administered to a patient can be determined by physical and physiological factors such as body weight, severity of condition, the type of disease being treated, previous or concurrent therapeutic interventions, idiopathy of the patient and on the route of administration. The practitioner of ordinary skill will rely on methods well established in the art to determine the concentration of active ingredient(s) in a composition and appropriate dose(s) for the individual subject.

In certain embodiments, pharmaceutical compositions may comprise, for example, at least about 0.1% of a protein or nucleic acid used according to the current invention. In other embodiments, a BST2 protein agent may comprise between about 2% to about 75% of the weight of the unit, or between about 25% to about 60%, for example, and any range derivable therein. In other non-limiting examples, a dose may also comprise from about 0.1 mg/kg/body weight to about 1000 mg/kg/body weight or any amount within this range, or any amount greater than 1000 mg/kg/body weight per administration.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, carbohydrates, sodium chloride or combinations thereof.

Sterile injectable solutions are prepared by incorporating a BST2 protein or nucleic acid encoding a BST2 protein in the required amount of the appropriate solvent with various amounts of the other ingredients enumerated above, as required, followed by filtered sterilization. Generally, dispersions are prepared by incorporating the various sterilized active ingredients into a sterile vehicle which contains the basic dispersion medium and/or the other ingredients.

In the case of sterile powders for the preparation of sterile injectable solutions, suspensions or emulsion, the preferred methods of preparation are vacuum-drying or freeze-drying or lyophilization techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In some embodiments, a Bst2 protein agent disclosed herein may be administered to the airways of a subject by any suitable means. In particular, a BST2 protein agent can be administered by generating an aerosol comprised of respirable particles, the respirable particles comprised of a BST2 protein agent, which particles the subject inhales. The respirable particles may be liquid or solid. The particles may optionally contain other therapeutic ingredients.

In an aspect, the present invention encompasses combination therapy for an autoimmune disease wherein a pharmaceutical composition comprises a BST2 protein agent, one or more pharmaceutically acceptable excipients and one or more other therapeutic agents. In some embodiments, a pharmaceutical composition comprising a BST2 protein agent and one or more other therapeutic agents can be used in accordance with a method of treating an autoimmune disease disclosed herein.

In preferred embodiments, a BST2 protein is comprised in a pharmaceutical composition with one or more other therapeutic agents such as immunosuppressive agents, anti-autoimmune agents, anti-inflammatory agents, steroids, corticosteroids, mucolytics, analgesics, anesthetics, adrenergic agents, antibiotics, surfactants, vitamins, moisturizing agents, and barrier-forming lipids. In an aspect, a pharmaceutical composition can comprise a BST2 protein agent, prednisone, and lipids in a liposomal formulation. In particularly preferred embodiments, a pharmaceutical composition of the present invention can comprise a BST2 protein agent, an anesthetic agent, prednisone and one or more pharmaceutically acceptable excipients.

In particular embodiments, prolonged absorption of an injectable composition can be brought about by the use in the compositions of agents delaying absorption, such as, for example, aluminum monostearate, gelatin or combinations thereof.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

### IX. Kits

Certain embodiments of the present invention are generally concerned with kits for treating autoimmune disorders, reducing interferon production in a cell, or for investigations of ILT7 binding and signaling behaviors.

In certain embodiments, the present invention provides a kit for treating an autoimmune disease or disorder comprising a container means, an injection means or an applicator means, a patient instruction means, and a pharmaceutical composition comprising a BST2 protein agent and one or more pharmaceutically acceptable excipients. The kit may also contain conventional pharmaceutical adjunct materials such as, for example, pharmaceutically acceptable salts to adjust the osmotic pressure, buffers, preservatives, antioxidants, and the like.

In particular embodiments, a kit for treating an autoimmune disease comprises a patient instruction means, a pharmaceutical composition comprising a BST2 protein agent, and an applicator means for topical or subcutaneous administration. In other embodiments, a kit for treating an autoimmune disease comprises a patient instruction means, a pharmaceutical composition comprising a BST2 protein agent, and an inhaler device for administration by inhalation. In still other embodiments, a kit for treating an autoimmune disease comprises a patient instruction means, a pharmaceutical composition comprising a BST2 protein agent, and an injection syringe or an intravenous infusion assembly.

When reagents and/or components comprising a kit are provided in a lyophilized form (lyophilisate) or as a dry powder, the lyophilisate or powder can be reconstituted by the addition of a suitable solvent. In particular embodiments, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

In select embodiments, kits are contemplated which comprise one or more BST2 protein agents, one or more container means, one or more reagents for modification of a BST2 protein agents, and/or one or more labeling or detection reagents. These kits may be used generally for investigating ILT7 receptor interactions and expression patterns, identifying an ILT7 ligand, and investigating ILT7 signaling events. In particular embodiments, the labeling or detection reagent is selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. In other embodiments, a kit is provided comprising one or more container means and a BST protein agent already labeled with a detection reagent selected from a group comprising a radioactive element, an enzyme, a molecule which absorbs light in the UV range, and a fluorophore.

As used herein the specification, “a” or “an” may mean one or more. As used herein in the claim(s), when used in conjunction with the word “comprising”, the words “a” or “an” may mean one or more than one. As used herein “another” may mean at least a second or more.

### EXAMPLES

The following examples are included to demonstrate preferred embodiments of the invention. It should be appreciated by those of skill in the art that the techniques disclosed in the following example represent techniques identified by the applicant to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### I. Example 1

### BST2 is an ILT7 Ligand

Here is presented the identification of BST2 as (1) a ligand of the ILT7 receptor on plasmacytoid dendritic cells (pDC), (2) a strong inhibitor, through its binding of ILT7, of type I interferon production by pDCs, and (3) a potentially therapeutic agent in the treatment of uncontrolled immune responses and/or autoimmunity.

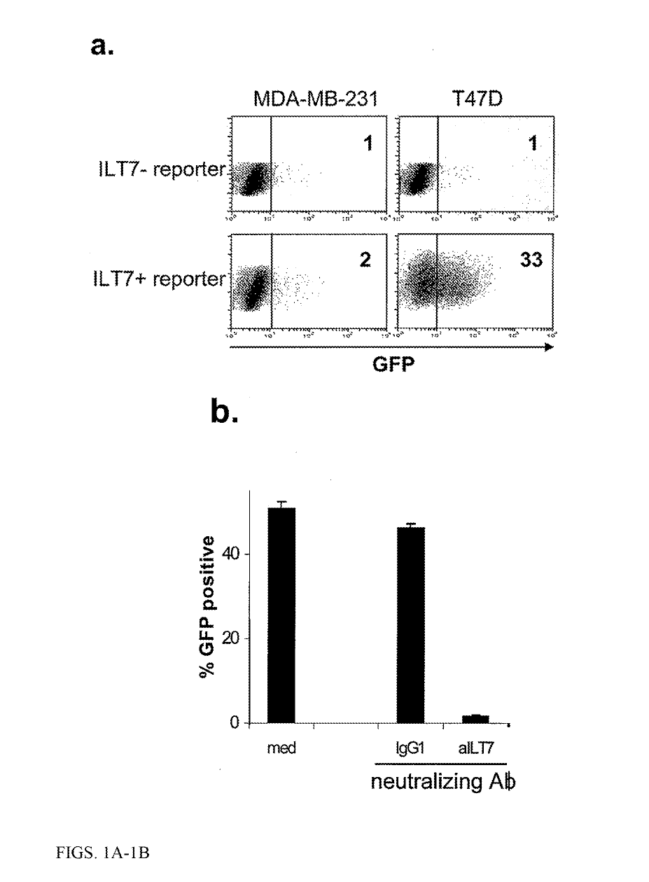

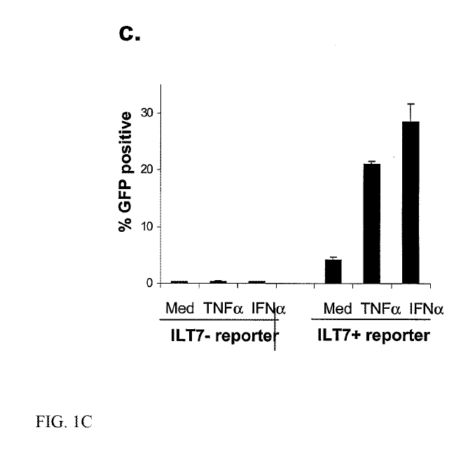

The NFAT-GFP reporter cell line expressing ILT7 and FcεRIγ (Cao et al., 2006) is used to search for ILT7 ligands on virus-infected cells and on a large panel of human tumor cell lines. Co-culture of the ILT7 reporter cells with human breast carcinoma T47D cells, but not with MDA-MB-231 cells, induced GFP expression (FIG. 1A). Human breast cancer MDA-MB-468 and MCF7 cell lines also activated the ILT7 reporter cells, whereas another breast cancer cell line, ZR-75-1, failed to do so (FIG. 5). Other transformed cell lines, such as HEK293, Vero, CHO, Cos7, and Jurkat, were also unable to stimulate the ILT7 reporter cells. The ability of T47D cells to activate the reporter cells is ILT7-dependent, because NFAT-GFP reporter cells expressing only the signaling adaptor FcεRIγ, and not ILT7, were not activated (FIG. 1A). In addition, the induction of GFP was completely abolished by a neutralizing anti-ILT7 mAb (FIG. 1B). Pre-treatment with IFNα and tumor necrosis factor (INF)α enhanced the ability of the breast cancer cells to activate the ILT7 reporters (FIG. 10), suggesting that ILT7-L expression is regulated by immune responses.

ILT7 activation by cancer cells requires direct cell-cell contact; cells cultured in separate chambers of a transwell dish fail to induce GFP. Although the two other ILT family members, ILT2 and ILT4, bind to the classical and non-classical MHC class I ligands (Brown et al., 2004; Navarro et al., 1999), the putative ILT7-L appears to be unrelated to MHC class I or class II, because antibodies against human MHC class I or class II did not block the ability of T47D cells to activate the ILT7 reporter cells and both the MHC class I-expressing cell lines (e.g., Jurkat and MDA-MB-231) and the MHC class II expression cell lines (e.g., EBV-transformed B cells) were unable to activate ILT7 reporter cells.

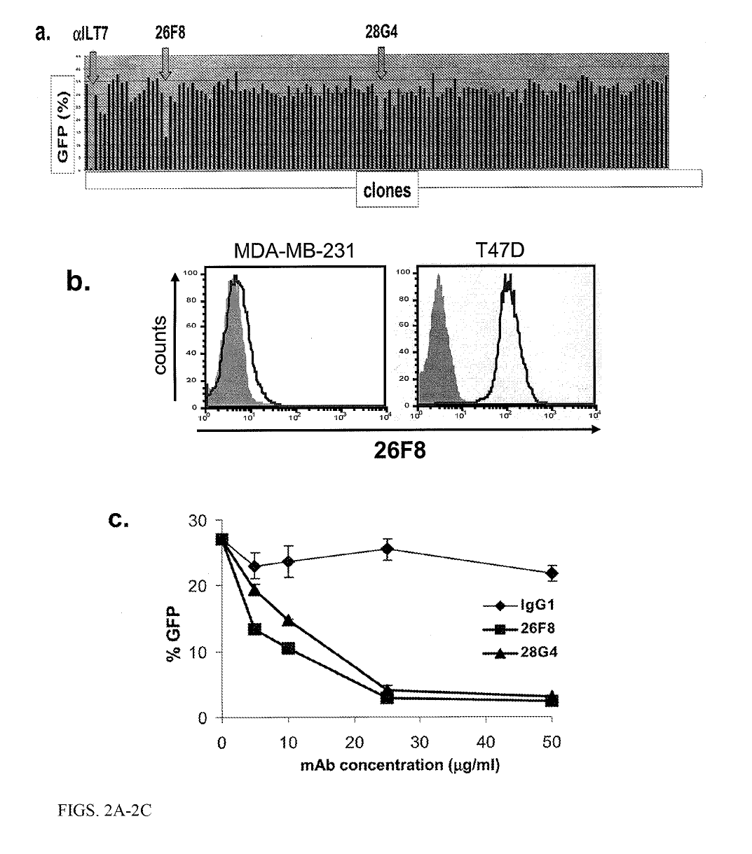

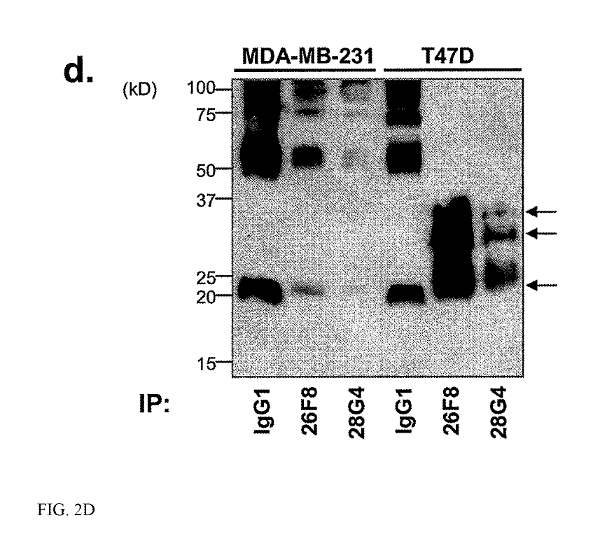

To identify the putative ILT7-L, BALB/c mice were immunized with T47D (ILT7-L^{pos}) and MDA-MB-231 (ILT7-L^{neg}) cells and obtained multiple hybridoma clones that bound specifically to T47D cells, but not to MDA-MB-231 cells. To identify mAbs that specifically recognize ILT7-L, the hybridoma clones were further screened for the ability to block T47D-induced ILT7 reporter cell activation. Two such clones (26F8 and 28G4) were identified (FIG. 2A). By using flow cytometry, it was determined that these two mAbs selectively stained the tumor cell lines T47D, MDA-MB-468, and MCF7, which activate ILT7 reporter cells (FIG. 2B; FIG. 5B), but not the cell lines HEK293, Vero, CHO, and Cos7, which fail to activate the ILT7 reporter cells. In addition, these two mAbs, but not the isotype-matched control mAb, strongly inhibited the ability of the tumor cell line T47D to activate the ILT7 reporter cells in a dose-dependent fashion (FIG. 2C). The two mAbs recognize non-overlapping epitopes, because one could not block the binding of the other to T47D cells, as determined by flow cytometry. Nevertheless, 26F8 and 28G4 mAbs immunoprecipitated three similar protein bands in T47D cells, but not in MDA-MB-231 cells (FIG. 2D), suggesting the presence of a cellular protein(s) as the potential ligand for ILT7.

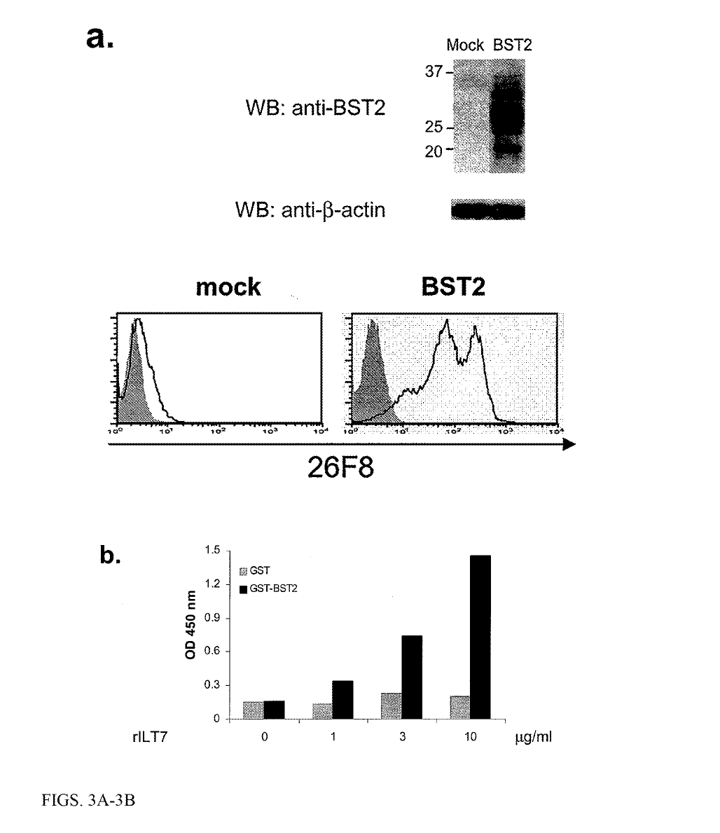

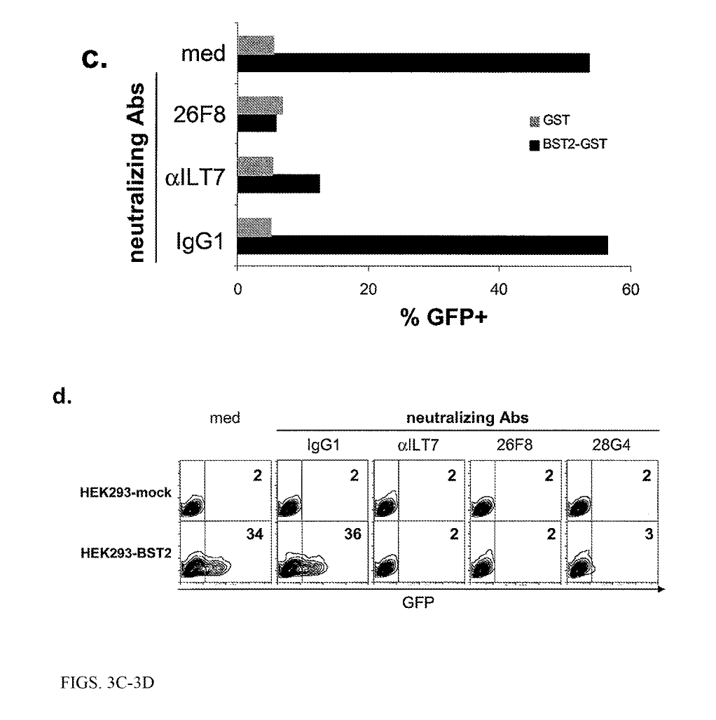

To identify the ligand for ILT7, 26F8 and 28G4 mAbs were used to screen a human cDNA library. Both antibodies specifically recognized the human bone marrow stromal cell antigen 2 (BST2; CD317). BST2 is a glycoprotein of 180 amino acids, which was initially identified as a membrane protein expressed by bone marrow stromal cells and later shown to be expressed by plasma cells and multiple types of cancer cells (Kupzig, 2003; Ohtomo et al., 1999; Walter-Yohrling et al., 2003). BST2 is expressed on many different types of cells after exposure to IFNα (Neil et al., 2008; Van Damme et al., 2008; Blasius et al., 2006). The 26F8 and 28G4 mAbs stained the human BST2 cDNA transfected HEK293 cells, but not parental cells by flow cytometry (FIG. 3A, right panel). In addition, recombinant ILT7 protein directly bound, a recombinant BST2-GST fusion protein, but not GST protein, in a dose-dependent manner (FIG. 3B). Furthermore, rBST2-GST fusion protein, but not GST alone, strongly activated the ILT7 reporter cells (FIG. 3C).

The specificity of the BST2-ILT7 interaction was demonstrated by the findings that: (1) rBST2-GST failed to induce GFP expression in ILT7-negative reporter cells, and (2) BST2-induced GFP expression in the ILT7 reporter cells was abrogated by neutralizing antibodies against either ILT7 or BST2, but not by control antibody. Furthermore, BST2 expressed on the surface of HEK293 cells induced GFP expression in ILT7 reporter cells, but not in ILT7-negative reporter cells. Again, activation of the ILT7 reporter was blocked by neutralizing antibodies against either ILT7 or BST2 (FIG. 3D). These data indicate BST2 as a biological ligand that specifically binds and activates ILT7.

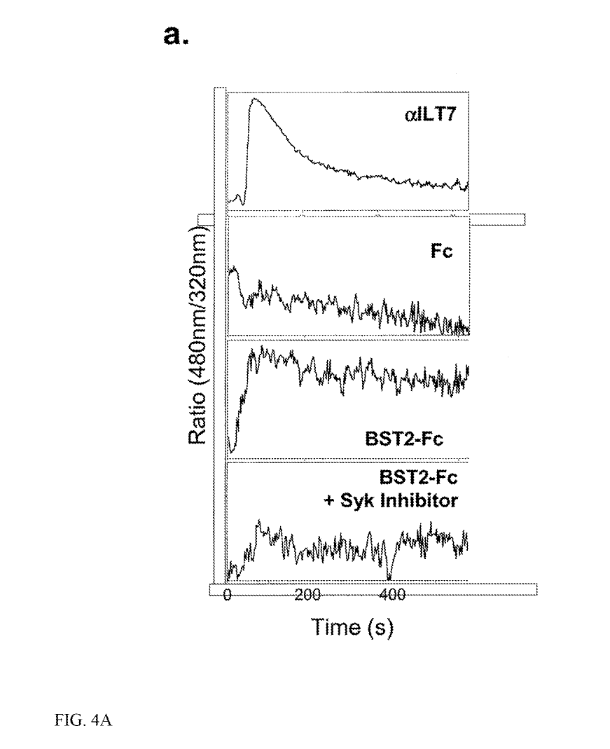

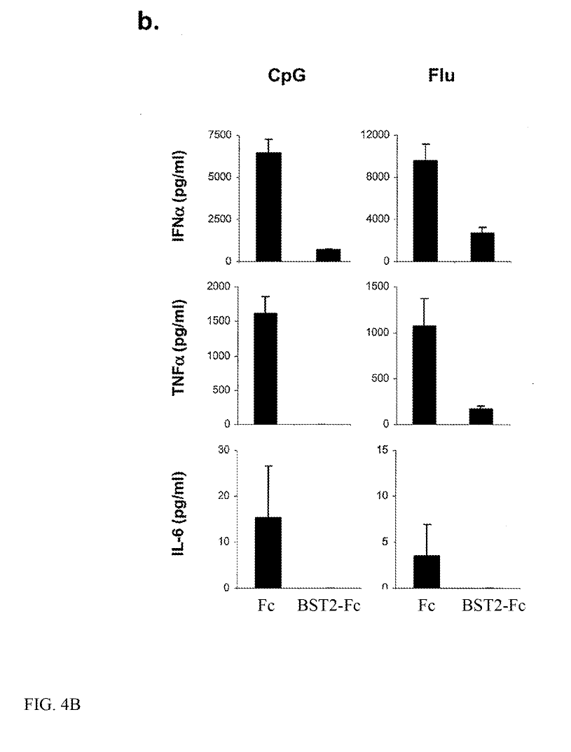

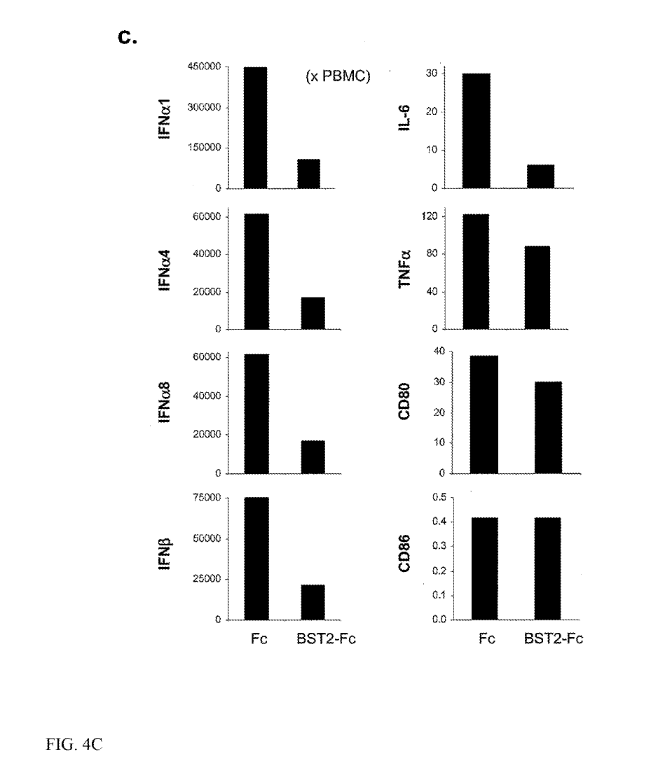

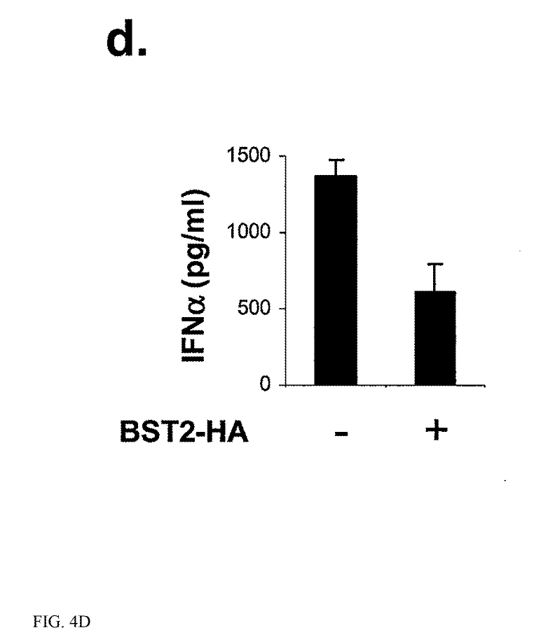

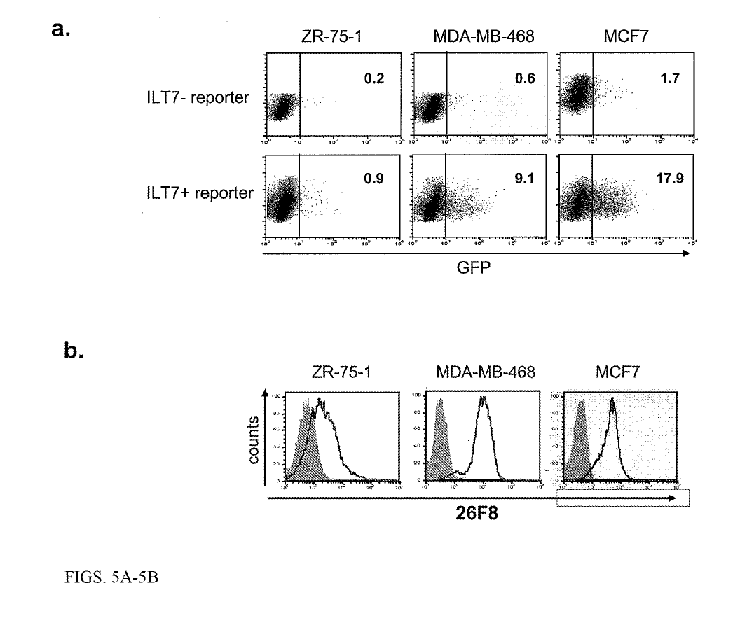

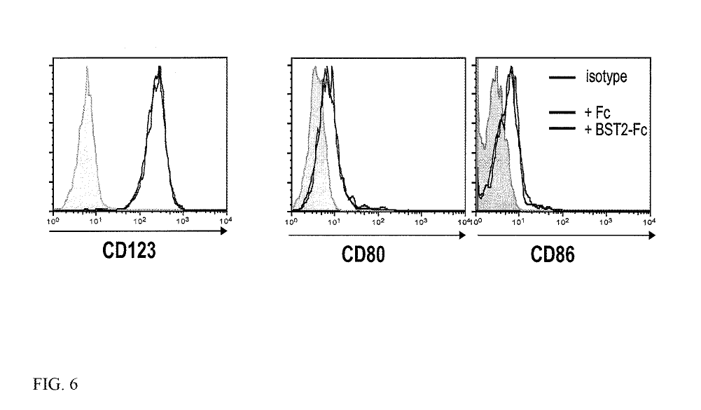

As shown previously, antibody crosslinking of ILT7 can induce prominent calcium influx in primary pDCs as a result of ITAM-mediated FcεRIγ signaling (Cao et al., 2006). Similarly, a rBST2-Fc protein, but not a control Fc protein alone, induced calcium mobilization in human pDCs, which depends on the function of Syk kinase (FIG. 4A). Since mAb crosslinking of ILT7 inhibits the immune response of pDCs, the effect of BST2 on TLR-induced cytokine responses by pDCs was investigated. Freshly isolated pDCs from human peripheral blood were pre-incubated with plate-bound rBST2-Fc protein or plate-bound Fc protein for 30 minutes, and were then challenged with influenza virus (Flu) or CpG, which trigger TLR7 and TLR9, respectively. The rBST2 protein suppressed the secretion of IFNα, INFα, and interleukin (IL)-6 (FIG. 4B), as well as the transcription of type I IFN subtypes, including IFNα1, IFNα4, IFNα8, and IFNI3 by pDCs (FIG. 4C) when activated by TLR ligands. In contrast, BST2 did not alter expression of costimulatory molecules, such as CD80 and CD86 by pDCs (FIG. 4C; FIG. 6C). Lastly, pDCs co-cultured with HEK293 cells expressing an HA-tagged BST2 secreted reduced levels of IFNα in response to Flu virus, when compared with pDCs in contact with untransfected HEK293 cells (FIG. 4D), suggesting that BST2-ILT7 interaction modulates pDCs' TLR-induced IFN responses.

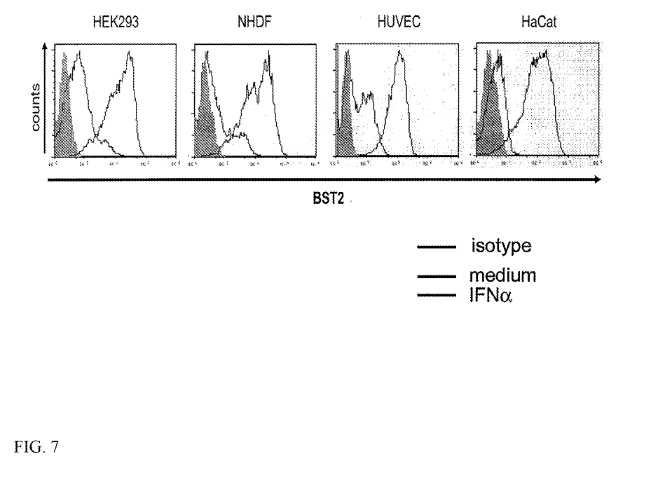

At this point, BST2 was identified as a biological ligand for a human pDC specific receptor ILT7. Interestingly, BST2 represents the first non-MHC class 1-type ligand for a member of the ILT receptor family (Brown et al., 2004). The pDCs play a critical role in anti-viral innate immune responses by secreting large quantities of IFNocip. However, the type I IFN responses immediately following viral infection are short-lived; if not, massive and prolonged IFN exposure will damage hematopoiesis, leading to lymphopenia (Kamphuis et al., 2006; Lin et al., 1998) and increase the risk of autoimmunity (Gota and Calabrese, 2003). Hence, a mechanism ensuring a specific and transient IFN response to viruses is critical to minimize the possibility of lymphopenia and autoimmune diseases in the host. As depicted in FIG. 7, BST2 is robustly induced on the surface of various types of cells following exposure to IFN and other proinflammatory cytokines, a consequence attributed to STAT activation (Ohtomo et al., 1999; Neil et al., 2008; Van Damme et al., 2008; Blasius et al., 2006). The BST2-ILT7 interaction, therefore, likely serves as an important negative feedback mechanism for preventing prolonged IFN production following viral infection.

Intracellular TLRs have limited ability to discriminate nucleic acids originated from host and foreign (Haas et al., 2008). Several host factors, including anti-DNA antibodies, anti-microbial peptide LL37, or the nuclear DNA-binding protein HMGB1, alone or in combination, facilitate entry of self-DNA into the endosomes of pDCs, where they trigger TLR9 to induce type I IFN responses (Blanco et al., 2001; Marshak-Rothstein and Rifkin, 2007; Tian et al., 2007). Similarly, autoantibody-self small nuclear ribonucleoprotein complexes can activate TLR7 through FcγRII to induce IFN (Vollmer et al., 2005; Savarese et al., 2006). This might lead to the constitutive activation of pDCs, which contributes to the autoimmune pathology of SLE and psoriasis. It will therefore be of interest to study if the BST2-ILT7-mediated controlling mechanism is breached under these conditions, which may provide an opportunity to develop therapeutics to down-modulate pDC activation during autoimmune conditions.

ILT7 Reporter Cell Assay

Unless otherwise noted, ELISA plates were coated overnight with mAbs at 10 μg/mL. Alternatively, 10⁵ cancer cells or transfected HEK293 cells were seeded in 24-well plates the day before the experiment. 10⁵ of ILT7⁺FcεRIγ or parental FcεRIγ⁺ 2B4 cells (Cao et al., 2006) were added to the ELISA plates or the cell monolayer. Plates were briefly spun at 100×g to pack the cells. After overnight culture, cells were subjected to flow cytometric analysis to measure GFP expression.

BST2 Activation of Human Primary pDCs

The institutional review board for human research at the M.D. Anderson Cancer Center approved the use of human blood samples for this study. Primary human pDCs were isolated from blood using a negative selection kit (Miltenyi Biotech) and sorted by flow cytometry as CD3−CD11c−CD14−CD15−CD16−CD19−CD56−CD4+CD123+ cells. pDCs were pre-incubated with plate-bound control Fc protein or BST2-Fc protein captured with 10 μg/mL of anti-human Fc F(ab)₂ (Jackson ImmunoResearch) on ELISA plate for 30 min prior to stimulation with 0.2 μM of CpG 2216 or inactivated influenza virus PR8 at MOI of 6. At 18 hrs later, the supernatants were harvested and analyzed for cytokines, and the cells were lysed and RNA subjected to RT-PCR analysis as described (Cao et al., 2006). To study calcium influx, purified control Fc protein or BST2-Fc protein was incubated with pDCs in the presence of goat anti-human Fc F(ab)₂ (Jackson ImmunoResearch). Dye loading and flow cytometric analysis was performed as described (Cao et al., 2006).

### Reagents and Cells

HEK293 cells were grown in high glucose Dulbecco's modified Eagle's medium (DMEM) supplemented with 10% fetal bovine serum, 50 U/mL penicillin, and 50 μg/mL streptomycin. Breast cancer cells and mouse 2B4 NFAT-GFP reporter cells (Cao et al., 2006) were grown in RPMI-1640 medium supplemented with 10% fetal bovine serum, 50 U/mL penicillin, and 50 μg/mL streptomycin. Normal human dermal fibroblasts (NHDF) and human umbilical vein endothelial cells (HUVEC) (Lonza) were cultured in Clonetics Fibroblast Cell Medium FGM-2 and Endothelial Cell Basal medium, respectively, and supplemented with growth factors following the manufacturer's recommendations. The human keratinocyte cell line HaCaT was kindly provided by Dr. Stephen Ullrich and cultured in RPMI-1640 medium supplemented with 10% fetal bovine serum, 50 U/mL penicillin, and 50 μg/mL streptomycin. TNFα was purchased from Peprotech; IFNα from Sigma.

### Anti-ILT7-L mAb Generation

Six- to eight-wk-old BALB/c mice were immunized with T47D and MDA-MB-231 cells by the alternate footpad method (Cao et al., 2006). Hybridoma clones secreting mAb that specifically stained T47D cells, but not MDA-MB-231 cells, were expanded. They were further screened for their ability to block T47D-induced GFP expression from ILT7+ reporter cells. mAbs 26F8 (IgG1) and 28G4 (IgG2a) were affinity-purified and fluorochrome-conjugated using mAb conjugation kits (Invitrogen).

cDNA Library Screening

A library of human full-length cDNA clones was purchased from OriGene Technologies, Inc. Plasmid DNA was prepared using the Wizard plus miniprep kit (Promega). For transfection, HEK293 cells were seeded at 10,000 cells/well in 96-well plates in 100 μL complete DMEM media and cultured overnight to reach 80% confluency. Fugene 6 (Roche) (0.6 μL) was added to 15 μL of Opti-MEM media (Invitrogen) and mixed with 10 μl of DNA in a 96-well plate. The mixture was incubated at room temperature for 15 minutes and then added to cells. Cells were incubated at 37° C. in 5% CO₂ for 48 hours.

For fluorometric microvolume assay technology (FMAT) detection, cells in 96-well plates were centrifuged at 200×g for 3 min, and the media were removed. mAbs 26F8 or 28G4 was added at 5 μg/mL, and mixed with Cy5-labeled goat anti-mouse IgG (Jackson ImmunoResearch). Cells were incubated at room temperature for 2 hours in the dark and then analyzed by using an FMAT 8100 HTS system (Applied Biosystems).

### BST2 Transfection and Expression Analysis

HEK293 cells were transfected with an expression plasmid containing the full-length human BST2 cDNA (Open Biosystems) with lipofectamine (Invitrogen). 48 hrs later, cells were either lysed for Western blot analysis using a BST2-specific rabbit polyclonal antibody (FabGennix, Inc.) or subjected to staining with fluorochrome-conjugated anti-ILT7-L mAbs. HEK293 cells stably expressing BST2-HA were transfected with pcDNA3-zeo-BST2-HA, selected with Zeocin and sorted for high HA expression by flow cytometry.

### Generation of Recombinant ILT7 and BST2 Fusion Protein

The extracellular domain of ILT7 or extracellular domain of BST2 (excluding the GPI anchor) were cloned into an expression vector containing a mutated human Fc fragment31. HEK293 cells were transiently transfected with the expression plasmids or the empty vector to produce recombinant protein, which was purified by using a Protein A column (GE Healthcare). The extracellular domain of BST2 (excluding the GPI anchor) was constructed as a GST-fusion protein, which was stably expressed in CHOK1SV cells and purified by glutathione Sepharose affinity chromatography (GE Healthcare).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,373,071
U.S. Pat. No. 4,401,796
U.S. Pat. No. 4,458,066
U.S. Pat. No. 4,598,049
U.S. Pat. No. 4,683,195
U.S. Pat. No. 4,683,202
U.S. Pat. No. 5,753,464
U.S. Pat. No. 5,914,252
U.S. Pat. No. 6,908,750
U.S. Pat. No. 6,921,751
U.S. Pat. No. 7,425,332
US Patent Appln. 2007/0212368
US Patent Appln. 2007/0154479
PCT Appln. WO 2006/037247
Animal Cell Culture, (Freshney), 1986.
Blanco et al., Cytokine Growth Factor Rev., 19:41-52, 2008.
Blanco et al., Science, 294:1540-1543, 2001.
Blasius et al., J. Immunol., 177:3260-3265, 2006.
Brown et al., Tissue Antigens, 64:215-225, 2004.
Cao et al., J. Exp. Med., 203:1399-1405, 2006.
Cao et al., PLoS Biology, 5:e248, 2007.
Chapman et al., Immunity, 11:603-613, 1999.
Chirgwin et al., Biochemistry, 18, 5294-5299, 1979.
Cho et al., Int. Immunol., 20:155-164, 2008.
Colonna et al., Nature Immunol., 5:1219-1226, 2004.
DNA Cloning: A Practical Approach,” Volumes I and II (Glover), 1985.
Eckert and Schmid, Arch. Dermatol., 125:1518-1524, 1989.
Gilliet et al., Nat. Rev. Immunol., 8:594-606, 2008.
Goeddel, Gene Expression Technology: Methods in Enzymology 185, Academic Press, San Diego, Calif., 1990.
Goeddel, Gene Expression Technology: Methods in Enzymology 185, Academic Press, San Diego, Calif., 1991.
Gota and Calabrese, Autoimmunity, 36:511-8, 2003.
Goto et al., Blood 1994; 84:1922-30, 1994.
Gulyas et al., Proc. Natl. Acad. Sci. USA, 92:10575-10579, 1995.
Haas et al., Immunity, 28:315-323, 2008.
Hartmann et al., Cancer Res., 63:6478-6487, 2003.
Honda and Taniguchi, Nat. Rev. Immunol., 6:644-658, 2006.
Hornung et al., J. Immunol., 168:4531-4537, 2002.
Houbenweyl, Methods of Organic Chemistry, Wansch (Ed.), Vol. 15 I and II, Thieme, Stuttgart, 1987.
Immobilized Cells And Enzymes, (IRL Press, 1986.
Ishikawa et al., Genomics, 26:527-34, 1995.
J. Biol. Chem., 243:3552-59, 1969.
Jarrossay et al., Eur. J. Immunol., 31:3388-3393, 2001.
Kadowaki et al., J. Exp. Med., 194:863-870, 2001.
Kamphuis et al., Blood, 108:3253-3261, 2006.
Kawai and Akira, Nat. Immunol., 7:131-137, 2006.
Kupzig, Traffic, 4:694-709, 2003.
Lande et al., Nature, 449:564-569, 2007.
Lin et al., J. Exp. Med., 187:79-87, 1998.
Maniatis et al., Molecular Cloning: A Laboratory Manual, 1982.
Marshak-Rothstein and Rifkin, Ann. Rev. Immun., 25:419-441, 2007.
McKenna et al. J. Virol., 79:17-27, 2005.
Merrifield, J. Am. Chem. Assoc., 85:2149-2154, 1964.
Miller et al., Biopolymers, 40:265-317, 1996.
Navarro et al., Eur. J. Immun., 29:277-283, 1999.
Neil et al., Nature, 451:425-430, 2008.
Nucleic Acid Hybridization, (Hames and Higgins), 1985.
O'Doherty et al., Immunology, 82:487-493, 1994.
Ohtomo et al., Biochem. Biophys. Res. Comm., 258:583-591, 1999.
Oligonucleotide Synthesis, (Gait), 1984.
Perbal, In: A Practical Guide To Molecular Cloning, 1984.
Queluz et al., J. Clin. Invest., 85(5): 1507-1515, 1990.
Remington's Pharmaceutical Sciences, 21th Ed. Lippincott Williams & Wilkins, 2005.
Rock et al., Eur. J. Immunol., 9999, NA, 2007.
Salio et al., Eur. J. Immunol., 33:1052-1062, 2003.
Sambrook et al., 1989
Savarese et al., Blood, 107:3229-3234, 2006.
Sorg et al., Blood, 93:2302-2307, 1999.
Stewart and Young, In: Solid Phase Peptide Synthesis; Pierce Chemical Co., Rockford, Ill., V: 176, 1984.
Tian et al., Nat. Immunol., 8:487-496, 2007.
Transcription and Translation, (Hames and Higgins) 1984.
Van Damme et al., Cell Host & Microbe., 3:245-252, 2008.
Vermi et al., J. Pathol., 200:255-268, 2003.
Vollmer et al., J. Exp. Med., 202:1575-1585, 2005.
Walter-Yohrling et al., Cancer Res., 63:8939-8947, 2003.
Wenzel et al., Exp. Dermat., 16:454-63, 2007.
Wollenberg et al., J. Investig. Dermatol., 119:1096-1102, 2002.
Yoneyama et al., J. Exp. Med., 195:1257-1266, 2002.
Zou et al., Nat. Med., 7:1339-1346, 2001.

1. A method of treating an autoimmune disease comprising
a) identifying a subject having or suspected of having an autoimmune disease, and
b) administering to the subject a therapeutically effective amount of a pharmaceutical composition comprising a BST2 protein agent
wherein the BST2 protein agent is capable of binding an ILT7 receptor and stimulating an ILT7 receptor response in a cell.

2. The method of claim 1, wherein the BST2 protein agent inhibits in said subject one or more aspects of an autoimmune response selected from the group consisting of production of type I interferon, production of autoantibodies, a mixed leukocyte reaction, a macrophage response, a natural killer reaction, and a lymphocyte activation.

3. The method of claim 1, wherein the BST2 protein agent is selected from the group consisting of a full length BST2 protein, a portion of a BST2 protein corresponding to one or more extracellular domains of a BST2 protein, a fragment of a BST2 protein, and a BST2 fusion protein.

4. The method of claim 1, wherein the BST2 protein agent is SEQ ID NO:1.

5. The method of claim 3, wherein the BST2 fusion protein comprises a full-length BST2 protein linked to an immunoglobin Fc region.

6. The method of claim 3, wherein the BST2 fusion protein comprises one or more extracellular domains of a BST2 protein linked to an immunoglobin Fc region.

7. The method of claim 3, wherein the BST2 fusion protein comprises a fragment of a BST2 protein linked to an immunoglobin Fc region, wherein said fragment is SEQ ID NO:2.

8. The method of claim 3, wherein the BST2 protein comprises an amino acid sequence having at least 95% sequence identity to SEQ ID NO:1.

9. The method of claim 3, wherein the BST2 protein comprises an amino acid sequence having at least 95% sequence identity to SEQ ID NO:2.

10. The method of claim 1, wherein the BST2 protein agent is administered in an amount which is sufficient to inhibit in a cell of the subject, one or more of type I interferon production, inflammatory cytokine production, and inflammatory chemokine production.

11. The method of claim 10, wherein type I interferon production is inhibited in a cell of the subject.

12. The method of claim 11, wherein the cell is a plasmacytoid dendritic cell.

13. The method of claim 1, wherein the autoimmune disease is selected from the group comprising systemic lupus erythematosus, cutaneous lupus erythematosus, Sjogren's syndrome, dermatomyositis, Goodpasture's syndrome, and psoriasis.

14. The method of claim 13, wherein the autoimmune disease is systemic lupus erythematosus.

15. The method of claim 13, wherein the autoimmune disease is cutaneous lupus erythematosus.

16. The method of claim 13, wherein the autoimmune disease is psoriasis.

17. The method of claim 1, wherein the pharmaceutical composition further comprises one or more pharmaceutically acceptable excipients.

18. The method of claim 1, wherein a BST2 protein agent is administered by one or more routes selected from the group comprising intravenously, subcutaneously, intramuscularly, intrasynovially, mucosally, topically, by inhalation, by subconjunctival injection, and by intraglandular injection.

19. A pharmaceutical composition comprising a BST2 protein agent in an amount effective to decrease interferon production in a patient.

20. The composition of claim 19, further comprises one or more pharmaceutically acceptable excipients.

21. The composition of claim 19, wherein the BST2 protein agent is selected from the group consisting of a full length BST2 protein, a portion of a BST2 protein corresponding to one or more extracellular domains of a BST2 protein, a fragment of a BST2 protein, and a BST2 fusion protein.

22. The composition of claim 19, wherein the BST2 protein agent comprises an amino acid sequence having at least 95% sequence identity to SEQ ID NO:1 or SEQ ID NO:2.

23. The composition of claim 19, further defined as a formulation suitable for administration by a route selected from the group comprising intravenously, subcutaneously, intramuscularly, intrasynovially, mucosally, topically, by inhalation, by subconjunctival injection, and by intraglandular injection


SEQUENCE LISTING (2 sequences)

SEQ ID NO: 1 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 2 - protein, 114 aa, Homo sapiens
NSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSS